(19) United States
(12) Patent Application Publication
(10) Pub. No.: US 2025/0364077 A1
(43) Pub. Date: Nov. 27, 2025

(54) GENERALIZED PROBABILISTIC GENERATIVE MODELING METHOD FOR ANALYSIS OF TUMOR METHYLATED MOLECULES IN TARGET CAPTURE REGIONS

(71) Applicant: GUARDANT HEALTH, INC., Palo Alto, CA (US)

(72) Inventors: Denis TOLKUNOV, Dublin, CA (US); Catalin BARBACIORU, Fremont, CA (US); Leylah DRUSBOSKY, Palm Harbor, FL (US)

(21) Appl. No.: 19/217,319

(22) Filed: May 23, 2025

Related U.S. Application Data
(60) Provisional application No. 63/651,101, filed on May 23, 2024.

(51) Int. Cl. G16B 20/20; G16B 40/20

(52) U.S. Cl. CPC G16B 20/20; G16B 40/20

(57) ABSTRACT

Disclosed herein are methods, compositions, and devices for use in diagnosis and treatment of cancer. The methods include a generative probabilistic accounting for the characteristics of methylation data, which includes random silencing and in possesses sparsity as a result. Here, the technique finds application in subtyping, determining disease transition and formation, among other oncology applications.

### CROSS-REFERENCE TO RELATED APPLICATION

The present application claims the benefit of U.S. Provisional Application No. 63/651,101, filed May 23, 2024, which is incorporated by reference in their entirety for all purposes.

### BACKGROUND

Small cell lung cancer (SCLC) transformation represents a mechanism of resistance to epidermal growth factor receptor tyrosine kinase inhibitors (EGFR-TKIs) in EGFR mutated non-small cell lung cancer (NSCLC) cases, which dramatically impacts patients' prognosis due to high refractoriness to conventional treatments.

SCLC transformation is one of the mechanisms of resistance to chemotherapy, immunotherapy, and targeted therapy in NSCLC. Two hypotheses have been used to explain the pathogenesis of SCLC transformation. Although SCLC transformation is not common in clinical practice, it has been repeatedly identified in many small patient series and case reports. It usually occurs in epidermal growth factor receptor (EGFR) mutant lung adenocarcinoma after treatment with tyrosine kinase inhibitors (TKIs). SCLC transformation can also occur in anaplastic lymphoma kinase (ALK)-positive lung cancer after treatment with ALK inhibitors and in wild-type EGFR or ALK NSCLC treated with immunotherapy. Chemotherapy was previously used to treat transformed SCLC, yet it is associated with an unsatisfactory prognosis SCLC transition are NSCLC patients treated samples that do not respond to TKI treatment

Lung cancer progression often involves tumors with heterogeneous histologies, harboring multiple subclones of distinct histological subtypes, lung adenocarcinomas (LUAD), lung squamous cell carcinomas (LUSC), SCLC. There is a need in the art for technologies to detect and characterize subtypes previously realized in histology. Described herein is a multi-modal, including genomie and epigenomic detection and analytical platform capable of capturing the partial contributions of subtype of each to the whole to enable assessment of mixed histology tumors, with further capabilities of detect small-cell transition.

### SUMMARY OF INVENTION

Described herein is a method, including: obtaining methylation data from a sample; generating a model based on the methylation data, wherein the model includes at least two parameters and a probabilistic distribution for each of a plurality of sites; transforming the methylation data based on the generated model; and determining at least one quantitative metric of the transformed methylation data. In various embodiments, the at least two parameters includes a molecule count, mixture component, or both. In various embodiments, the molecule count includes a region score. In various embodiments, the mixture component includes a measurement of tumor and normal molecules. In various embodiments, the mixture component including a measurement of tumor and normal molecules includes fitting. In various embodiments, fitting includes parametric methods, regression, composite distribution, among others. In various embodiments, the probabilistic distribution includes a normal, Poisson, Bayesian inference, Bernoulli distribution. In various embodiments, the methylation data is generated by detecting methylation in at least one of a plurality of sites. In various embodiments, the plurality of sites are obtained from a sample. In various embodiments, determining at least one quantitative metric of the transformed methylation data characterizes a sample. In various embodiments, charactering the sample includes determining the sample is derived from one or more subtypes. In various embodiments, the one or more subtypes are selected from the group consisting of: lung adenocarcinomas (LUAD), lung squamous cell carcinomas (LUSC), small cell lung cancer (SCLC) and/or non-small cell lung cancer (NSCLC). In various embodiments, the pone or more subtypes are selected from the group consisting of: HR, HER2, and TNBC. In various embodiments, characterizing the sample includes determining transition, transformation or other alteration of a cancer disease phenotype. In various embodiments, the method includes obtaining a sample. In various embodiments, the method includes having obtained a sample. In various embodiments, the method includes having recommending and/or selecting a treatment based on the characterization of the sample. In various embodiments, the method includes having administering a treatment based on the characterization of the sample. In various embodiments, the model includes one or more of Equation 1, 2, 3, 4, 5, 6, 7, and 8. In various embodiments, the treatment includes one or more therapeutic agents selected from the group consisting of: cisplatin, carboplatin, gemcitabine, taxanes, pemetrexed, VEGFR inhibitor, optionally including bevacizumab, or EGFR inhibitor, optionally including erlotinib. In various embodiments, the sample includes cell-free DNA. In various embodiments, the method includes diagnosing a subject. In various embodiments, the method includes prognosing a subject for one or more outcomes.

A system configured to perform a method, including: obtaining methylation data from a sample; generating a model based on the methylation data, wherein the model includes at least two parameters and a probabilistic distribution for each of a plurality of sites; transforming the methylation data based on the generated model; and determining at least one quantitative metric of the transformed methylation data. In various embodiments, the at least two parameters includes a molecule count, mixture component, or both. In various embodiments, the molecule count includes a region score. In various embodiments, the mixture component includes a measurement of tumor and normal molecules. In various embodiments, the mixture component including a measurement of tumor and normal molecules includes fitting. In various embodiments, fitting includes parametric methods, regression, composite distribution, among others. In various embodiments, the probabilistic distribution includes a normal, Poisson, Bayesian inference, Bernoulli distribution. In various embodiments, the probabilistic distribution includes a Bernoulli distribution. In various embodiments, the methylation data is generated by detecting methylation in at least one of a plurality of sites. In various embodiments, the plurality of sites are obtained from a sample. In various embodiments, determining at least one quantitative metric of the transformed methylation data characterizes a sample. In various embodiments, charactering the sample includes determining the sample is derived from one or more subtypes. In various embodiments, the one or more subtypes are selected from the group consisting of: lung adenocarcinomas (LUAD), lung squamous cell carcinomas (LUSC), small cell lung cancer (SCLC) and/or non-small cell lung cancer (NSCLC). In various embodiments, the pone or more subtypes are selected from the group consisting of: HR, HER2, and TNBC. In various embodiments, characterizing the sample includes determining transition, transformation or other alteration of a cancer disease phenotype. In various embodiments, the method includes obtaining a sample. In various embodiments, the method includes having obtained a sample. In various embodiments, the method includes having recommending and/or selecting a treatment based on the characterization of the sample. In various embodiments, the method includes having administering a treatment based on the characterization of the sample. In various embodiments, the model includes one or more of Equation 1, 2, 3, 4, 5, 6, 7, and 8. In various embodiments, the treatment includes one or more therapeutic agents selected from the group consisting of: cisplatin, carboplatin, gemcitabine, taxanes, pemetrexed, VEGFR inhibitor, optionally including bevacizumab, or EGFR inhibitor, optionally including erlotinib. In various embodiments, the sample includes cell-free DNA. In various embodiments, the method includes diagnosing a subject. In various embodiments, the method includes prognosing a subject for one or more outcomes.

A computer readable medium for performing a method, including: obtaining methylation data from a sample; generating a model based on the methylation data, wherein the model includes at least two parameters and a probabilistic distribution for each of a plurality of sites; transforming the methylation data based on the generated model; and determining at least one quantitative metric of the transformed methylation data. In various embodiments, the at least two parameters includes a molecule count, mixture component, or both. In various embodiments, the molecule count includes a region score. In various embodiments, the mixture component includes a measurement of tumor and normal molecules. In various embodiments, the mixture component including a measurement of tumor and normal molecules includes fitting. In various embodiments, fitting includes parametric methods, regression, composite distribution, among others. In various embodiments, the probabilistic distribution includes a normal, Poisson, Bayesian inference, Bernoulli distribution. In various embodiments, the probabilistic distribution includes a Bernoulli distribution. In various embodiments, the methylation data is generated by detecting methylation in at least one of a plurality of sites. In various embodiments, the plurality of sites are obtained from a sample. In various embodiments, determining at least one quantitative metric of the transformed methylation data characterizes a sample. In various embodiments, charactering the sample includes determining the sample is derived from one or more subtypes. In various embodiments, the one or more subtypes are selected from the group consisting of: lung adenocarcinomas (LUAD), lung squamous cell carcinomas (LUSC), small cell lung cancer (SCLC) and/or non-small cell lung cancer (NSCLC). In various embodiments, the pone or more subtypes are selected from the group consisting of: HR, HER2, and TNBC. In various embodiments, characterizing the sample includes determining transition, transformation or other alteration of a cancer disease phenotype. In various embodiments, the method includes obtaining a sample. In various embodiments, the method includes having obtained a sample. In various embodiments, the method includes having recommending and/or selecting a treatment based on the characterization of the sample. In various embodiments, the method includes having administering a treatment based on the characterization of the sample. In various embodiments, the model includes one or more of Equation 1, 2, 3, 4, 5, 6, 7, and 8. In various embodiments, the treatment includes one or more therapeutic agents selected from the group consisting of: cisplatin, carboplatin, gemcitabine, taxanes, pemetrexed, VEGFR inhibitor, optionally including bevacizumab, or EGFR inhibitor, optionally including erlotinib. In various embodiments, the sample includes cell-free DNA. In various embodiments, the method includes diagnosing a subject. In various embodiments, the method includes prognosing a subject for one or more outcomes.

In various embodiments, the least one quantitative metric includes a methylation profile for at least one nucleic acid sequence obtained from a human subject; and selecting a treatment suitable for the human subject based on the methylation profile. In other embodiments, the methylation profile includes at least one differentially menthylated region (DMR) In other embodiments, the at least one DMR is determined based on a comparison to a threshold determined from one or more healthy subjects.

In various embodiment, the methylation profile is used to determine that a patient is afflicted with a lung cancer subtype. In various embodiments, the lung cancer subtype is one or more subtypes selected from the group consisting of: lung adenocarcinomas (LUAD), lung squamous cell carcinomas (LUSC), small cell lung cancer (SCLC) and/or non-small cell lung cancer (NSCLC). In various embodiment, the determination includes application of equation 1 and/or 2.

In various embodiments, the methylation profile is detected using a methyl binding domain (MBD) partitioning assay. In other embodiments, the MBD partitioning assay includes combining a plurality of nucleic acid molecules derived from the human subject with a solution including an amount of methyl binding domain (MBD) proteins to produce a nucleic acid-MBD protein solution; and performing a plurality of washes of the nucleic acid-MBD protein solution with a salt solution to produce a number of nucleic acid fractions, individual nucleic acid fractions having a threshold number of methylated cytosines in regions of the plurality of nucleic acids having at least the threshold cytosine-guanine content. In various embodiments, the treatment includes one or more therapeutic agents selected from the group consisting of: cisplatin, carboplatin, gemcitabine, taxanes, pemetrexed, VEGFR inhibitor, optionally including bevacizumab, or EGFR inhibitor, optionally including erlotinib. In various embodiments, the sample includes cell-free DNA. In various embodiments, the selection of treatment is based on a determination that a patient is afflicted with lung adenocarcinomas (LUAD), lung squamous cell carcinomas (LUSC), small cell lung cancer (SCLC) and/or non-small cell lung cancer (NSCLC).

In various embodiments, the method includes applying the at least one quantitative metric to generate a methylation profile for at least one nucleic acid sequence obtained from a human subject. In various embodiments, the method includes selecting a treatment suitable for the human subject based on the methylation profile using a database, wherein the database includes a plurality of nucleic acid sequence information, and methylation status from a plurality of subjects and identifying from the plurality of subjects with matching genetic and epigenetic information, prior treatment of the plurality of subjects with matching genetic information.

In various embodiments, the method includes applying the at least one quantitative metric to determining a state of biological molecules obtained from a sample derived from a human subject, and detecting biological molecules in the sample. In other embodiments, the biological molecules are one or more of: DNA, methylated DNA, RNA, methylated RNA, proteins, and peptides. In other embodiments, the method includes testing combining a plurality of nucleic acid molecules derived from a subject with a solution including an amount of methyl binding domain (MBD) proteins to produce a nucleic acid-MBD protein solution; and performing a plurality of washes of the nucleic acid-MBD protein solution with a salt solution to produce a number of nucleic acid fractions, individual nucleic acid fractions having a threshold number of methylated cytosines in regions of the plurality of nucleic acids having at least the threshold cytosine-guanine content. In other embodiments, the wash of the plurality of washes is performed with a solution having a concentration of sodium chloride (NaCl) and produces a nucleic acid fraction of the number of nucleic acid fractions having a range of binding strengths to MBD proteins. In other embodiments, the method includes determining that a first nucleic acid fraction is associated with a first partition of a plurality of partitions of nucleic acids, the first partition corresponding to a first range of binding strengths to MBD proteins, attaching a first molecular barcode to nucleic acids of the first nucleic acid fraction, the first molecular barcode being included in a first set of molecular barcodes associated with the first partition, determining that a second nucleic acid fraction is associated with a second partition of the plurality of partitions of nucleic acids, the second partition corresponding to a second range of binding energies to MBD proteins different from the first range of binding strengths to MBD proteins, and attaching a second molecular barcode to nucleic acids of the second nucleic acid fraction, the second molecular barcode being included in a second set of molecular barcodes associated with the second partition. In other embodiments, the method includes combining at least a portion of the number of nucleic acid fractions with an amount of restriction enzyme that cleaves molecules with one or more unmethylated cytosines to produce at least a portion of the plurality of samples used to produce the sequencing reads, wherein the threshold amount of methylated cytosines corresponds to a minimum frequency of methylated cytosines within a region having at least the threshold cytosine-guanine content.

### BRIEF DESCRIPTION OF FIGURES

FIG. 1. Modeling tumor molecules with probabilistic generative model. As described, methylation data is sparse and signal correlates with the DNA input and TF. To take this biological activity into account for improving the accuracy of methylation measurements, a probabilistic model can take into account a region score (molecule count in a region) that depends on the TF, incorporate the parameters of observed molecules that are a mixture of normal molecules (background) and tumor molecules as s mixture component (Equation 2). One then combines both the mixture component involving normal and tumor molecules as features, and a Bernoulli distribution to account for probabilistic testing of sporadic silencing and activation of methylatio (Equation 1), as shown.

FIG. 2. Probabilistic generative model. A further expanded illustration of the model of FIG. 1, including Bernoulli distribution (Equation 1) and mixture model (Equation 2).

FIG. 3. Samples selection for a probabilistic model. As described herein, from 23,869 samples, sub-sets included cancer free samples (No non-synonymous SNV/Indels in reportable genes), tumor BRCA samples (found to have non-synonymous SNVs/Indels in reportable genes).

FIG. 4. Representative applications Predictions of the mixture component of the model Predictions of the Bernoulli component of the model. Various examples of applications include measuring tumor fraction by inferring unknown tumor fraction as a latent variable (Equation 4), normalized methylation score (for cancer subtyping) by using the probability of tumor molecules present in a genomic region given a region score (Equation 5), tumor fraction and DNA input of a sample, or predicting the likelihood of different cancer subtypes (Equation 6) with each shown in FIG. 4. Performance characteristics are shown in FIG. 4A mixture component, FIG. 4B Bernoulli for characterizing FIG. 4C BRCA samples. Results are drawn from 8,000 in silico dilution samples and ˜2,400 BRCA regions for BRCA samples.

FIG. 5. Probability of tumor molecules present in a genomic region. Determining probability of tumor molecules present in a genomic region, begins with region score (Equation 7) followed by fitting of background distribution to normal cancer free samples, then fitting the mixture to tumor BRCA samples (Equation 2). Thereafter, a test sample can be evaluated for the probability of tumor molecules in the region given the DNA input (total pos. cntrl.) and tumor fraction, having fitted distributions between normal and cancer samples (Equation 8). Here, the Inventors used molecular counts in 15,229 promoter regions for region score.

FIGS. 6A and 6B. Exploring the mixture distribution component of the model. In this example, the distribution of tumor molecules in region I is modeled as Student's t-distribution. Region scores across 1,014 BRCA samples, FIG. 6A is raw molecule counts, then fitted mixture model, and FIG. 6B depicts region scores across various MAFs.

FIGS. 7A-7C. Exploring the mixture distribution component of the model. Region scores across 1,014 BRCA samples. FIG. 7A is raw molecule counts, then fitted mixture model, and FIG. 7B depicts region scores across various MAFs. As shown in FIG. 7C, optionally, one can apply a filter to exclude the regions where the fit quality is poor.

FIG. 8. Probabilistic modeling of methylation signal. For performance comparison, described are two approaches to BRCA subtyping. A first approach includes transferring the subtype prediction models between different technologies to train three models (HR/HER2/TNBC) using 450K methylation calls data from TCGA primary tissue samples and applying these subtype prediction models to methylation calls for subtype prediction. A second approach is to training the models (LR+Lasso) directly on epigenomic methylation data (samples with known subtypes) and evaluate the performance using cross validation.

FIGS. 9A and 9B. Epigenomic breast cancer subtyping results. HR & TNBC classifiers trained on TCGA meth calls from primary tumors, with cohorts drawn from Tables 1 and 2 . . . . HER2 classifier trained on epigenomic samples with TF>0.01 using 5-fold CV. In FIG. 9A and FIG. 9B as shown, the described generative probabilistic method provides excellent determination of subtype accuracy, at least comparable and in some instances superior to direct training on sample followed by cross validation.

FIGS. 10A and 10B. SCLC prediction model. Here, shown in FIG. 10A is 10-fold CV of logistic regression with lasso regularization. The 235 NSCLC and 87 SCLC samples with TF>0.1% are split into 10 bins and cross validation is used to predict SCLC score for all 322 samples using logistic regression (LR) with lasso regularisation. Resulting scores separate well between NSCLC (SCLC neg) and SCLC patients and shown in FIG. 10B are ROC curves and AUC.

FIG. 11. SCLC transformation performance characteristics. As shown no correlation between score and TF for NSCLC patients (red). This is expected IF the prediction model is picking biological differences between NSCLC and SCLC and not if it is ‘driven” by TF instead, thereby demonstrating true signal determination.

### DETAILED DESCRIPTION

While various embodiments of the disclosure have been shown and described herein, those skilled in the art will understand that such embodiments are provided by way of example only. Numerous variations, changes, and substitutions may occur to those skilled in the art without departing from the disclosure. It should be understood that various alternatives to the embodiments of the disclosure described herein may be employed.

The term “about” and its grammatical equivalents in relation to a reference numerical value can include a range of values up to plus or minus 10% from that value. For example, the amount “about 10” can include amounts from 9 to 11. The term “about” in relation to a reference numerical value can include a range of values plus or minus 10%, 9%, 8%, 7%, 6%, 5%, 4%, 3%, 2%, or 1% from that value.

The term “at least” and its grammatical equivalents in relation to a reference numerical value can include the reference numerical value and greater than that value. For example, the amount “at least 10” can include the value 10 and any numerical value above 10, such as 11, 100, and 1,000.

The term “at most” and its grammatical equivalents in relation to a reference numerical value can include the reference numerical value and less than that value. For example, the amount “at most 10” can include the value 10 and any numerical value under 10, such as 9, 8, 5, 1, 0.5, and 0.1.

As used herein the singular forms “a”, “an”, and “the” can include plural referents unless the context clearly dictates otherwise. Thus, for example, reference to “a cell” can include a plurality of such cells and reference to “the culture” can include reference to one or more cultures and equivalents thereof known to those skilled in the art, and so forth. All technical and scientific terms used herein can have the same meaning as commonly understood to one of ordinary skill in the art to which this disclosure belongs unless clearly indicated otherwise.

Current approaches are to omit testing both genomic and epigenomic attributes of the patient sample or to perform multiple tests separately. Omitting genomic or epigenomic information can result in prescription of cancer therapies that could be known to be ineffective or withholding cancer therapies that could be known to be effective, had both genomic and epigenomic information been available. Cancer can be indicated by epigenetic variations, such as methylation. Examples of methylation changes in cancer include local gains of DNA methylation in the CpG islands at the transcription start site (TSS) of genes involved in normal growth control, DNA repair, cell cycle regulation, and/or cell differentiation. This hypermethylation can be associated with an aberrant loss of transcriptional capacity of involved genes and occurs at least as frequently as point mutations and deletions as a cause of altered gene expression. DNA methylation profiling can be used to detect regions with different extents of methylation (“differentially methylated regions” or “DMRs”) of the genome that are altered during development or that are perturbed by disease, for example, cancer or any cancer-associated disease. The genome of cancer cells harbor imbalance in the above DNA methylation patterns, and therefore in functional packaging of the DNA. The abnormalities of chromatin organization are therefore coupled with methylation changes and may contribute to enhanced cancer profiling when analyzed jointly. Combining MBD-partitioning with fragmentomic data, such as fragment mapped starts and stops positions (correlated with nucleosome positions), fragment length and associated nucleosome occupancy, can be used for chromatin structure analysis in hypermethylation studies with the aim to improve biomarker detection rate.

Methylation profiling can involve determining methylation patterns across different regions of the genome. For example, after partitioning molecules based on extent of methylation (e.g., relative number of methylated sites per molecule) and sequencing, the sequences of molecules in the different partitions can be mapped to a reference genome. This can show regions of the genome that, compared with other regions, are more highly methylated or are less highly methylated. In this way, genomic regions, in contrast to individual molecules, may differ in their extent of methylation.

A characteristic of nucleic acid molecules may be a modification, which may include various chemical or protein modifications (i.e. epigenetic modifications). Non-limiting examples of chemical modification may include, but are not limited to, covalent DNA modifications, including DNA methylation. In some embodiments, DNA methylation includes addition of a methyl group to a cytosine at a CpG site (a cytosine followed by a guanine in a nucleic acid sequence). In some embodiments, DNA methylation includes addition of a methyl group to adenine, such as in N6-methyladenine. In some embodiments, DNA methylation is 5-methylation (modification of the 5th carbon of the 6 carbon ring of cytosine). In some embodiments, 5-methylation includes addition of a methyl group to the 5C position of the cytosine to create 5-methylcytosine (m5c). In some embodiments, methylation includes a derivative of m5c. Derivatives of m5c include, but are not limited to, 5-hydroxymethylcytosine (5-hmC), 5-formylcytosine (5-fC), and 5-caryboxylcytosine (5-caC). In some embodiments, DNA methylation is 3C methylation (modification of the 3rd carbon of the 6 carbon ring of cytosine). In some embodiments, 3C methylation includes addition of a methyl group to the 3C position of the cytosine to generate 3-methylcytosine (3mC). Other examples include N6-methyladenine or glycosylation. DNA methylation includes addition of methyl groups to DNA (e.g. CpG) and can change the expression of methylated DNA region. Methylation can also occur at non CpG sites, for example, methylation can occur at a CpA, CpT, or CpC site. DNA methylation can change the activity of methylated DNA region. For example, when DNA in a promoter region is methylated, transcription of the gene may be repressed. DNA methylation is critical for normal development and abnormality in methylation may disrupt epigenetic regulation. The disruption, e.g., repression, in epigenetic regulation may cause diseases, such as cancer. Promoter methylation in DNA may be indicative of cancer.

A CpG dyad is the dinucleotide CpG (cytosine-phosphate-guanine, i.e. a cytosine followed by a guanine in a 5′→3′ direction of the nucleic acid sequence) on the sense strand and its complementary CpG on the antisense strand of a double-stranded DNA molecule. CpG dyads can be either fully methylated or hemi-methylated (methylated on one strand only).

The CpG dinucleotide is underrepresented in the normal human genome, with the majority of CpG dinucleotide sequences being transcriptionally inert (e.g. DNA heterochromatic regions in pericentromeric parts of the chromosome and in repeat elements) and methylated. However, many CpG islands are protected from such methylation especially around transcription start sites (TSS).

Protein modifications include binding to components of chromatin, particularly histones including modified forms thereof, and binding to other proteins, such as proteins involved in replication or transcription. The disclosure provides methods of processing and analyzing nucleic acids with different extents of modification, such that the nature of their original modification is correlated with a nucleic acid tag and can be decoded by sequencing the tag when nucleic acids are analyzed. Genetic variation of sample nucleic acid modifications can then be associated with the extent of modification (epigenetic variation) of that nucleic acid in the original sample, include single stranded (e.g., ssDNA or RNA) or double stranded molecules (e.g., dsDNA).

The loss of DNA can reduce the presence of one or more types of DNA such that the presence of the one or more types of DNA such as cfDNA, is difficult to detect. In one or more additional scenarios, existing methods to measure DNA methylation, such as enrichment or depletion methods, can have a relatively high level of resolution, such as about 100 base pairs (bp) to about 200 bp that can make accurately determining an amount of methylation of DNA difficult. The accuracy with which DNA methylation is determined can impact the accuracy of estimates of tumor fraction for samples. Since tumor fraction can be used to determine whether a sample is derived from a subject in which a tumor is present or not, the accuracy of determinations of tumor fraction estimates can impact diagnosis and/or treatment decisions for individuals.

### Samples

A sample can be any biological sample isolated from a subject. A sample can be a bodily sample. Samples can include body tissues, such as known or suspected solid tumors, whole blood, platelets, serum, plasma, stool, red blood cells, white blood cells or leucocytes, endothelial cells, tissue biopsies, cerebrospinal fluid synovial fluid, lymphatic fluid, ascites fluid, interstitial or extracellular fluid, the fluid in spaces between cells, including gingival crevicular fluid, bone marrow, pleural effusions, cerebrospinal fluid, saliva, mucous, sputum, semen, sweat, urine. Samples are preferably body fluids, particularly blood and fractions thereof, and urine. A sample can be in the form originally isolated from a subject or can have been subjected to further processing to remove or add components, such as cells, or enrich for one component relative to another. Thus, a preferred body fluid for analysis is plasma or serum containing cell-free nucleic acids. A sample can be isolated or obtained from a subject and transported to a site of sample analysis. The sample may be preserved and shipped at a desirable temperature, e.g., room temperature, 4° C., −20° C., and/or −80° C. A sample can be isolated or obtained from a subject at the site of the sample analysis. The subject can be a human, a mammal, an animal, a companion animal, a service animal, or a pet. The subject may have a cancer. The subject may not have cancer or a detectable cancer symptom. The subject may have been treated with one or more cancer therapy, e.g., any one or more of chemotherapies, antibodies, vaccines or biologics. The subject may be in remission. The subject may or may not be diagnosed as being susceptible to cancer or any cancer-associated genetic mutations/disorders.

The volume of plasma can depend on the desired read depth for sequenced regions. Exemplary volumes are 0.4-40 ml, 5-20 ml, 10-20 ml. For example, the volume can be 0.5 mL, 1 mL, 5 mL 10 mL, 20 mL, 30 mL, or 40 mL. The volume of sampled plasma may be 5 to 20 mL.

A sample can comprise various amounts of nucleic acid that contains genome equivalents. For example, a sample of about 30 ng DNA can contain about 10,000 (104) haploid human genome equivalents and, in the case of cfDNA, about 200 billion (2×1011) individual polynucleotide molecules. Similarly, a sample of about 100 ng of DNA can contain about 30,000 haploid human genome equivalents and, in the case of cfDNA, about 600 billion individual molecules.

A sample can comprise nucleic acids from different sources, e.g., from cells and cell-free of the same subject, from cells and cell-free of different subjects. A sample can comprise nucleic acids carrying mutations. For example, a sample can comprise DNA carrying germline mutations and/or somatic mutations. Germline mutations refer to mutations existing in germline DNA of a subject. Somatic mutations refer to mutations originating in somatic cells of a subject, e.g., cancer cells. A sample can comprise DNA carrying cancer-associated mutations (e.g., cancer-associated somatic mutations). A sample can comprise an epigenetic variant (i.e. a chemical or protein modification), wherein the epigenetic variant is associated with the presence of a genetic variant such as a cancer-associated mutation. In some embodiments, the sample includes an epigenetic variant associated with the presence of a genetic variant, wherein the sample does not comprise the genetic variant.

Exemplary amounts of cell-free nucleic acids in a sample before amplification range from about 1 fg to about 1 μg, e.g., 1 μg to 200 ng, 1 ng to 100 ng, 10 ng to 1000 ng. For example, the amount can be up to about 600 ng, up to about 500 ng, up to about 400 ng, up to about 300 ng, up to about 200 ng, up to about 100 ng, up to about 50 ng, or up to about 20 ng of cell-free nucleic acid molecules. The amount can be at least 1 fg, at least 10 fg, at least 100 fg, at least 1 pg, at least 10 pg, at least 100 pg, at least 1 ng, at least 10 ng, at least 100 ng, at least 150 ng, or at least 200 ng of cell-free nucleic acid molecules. The amount can be up to 1 femtogram (fg), 10 fg, 100 fg, 1 picogram (pg), 10 μg, 100 pg, 1 ng, 10 ng, 100 ng, 150 ng, or 200 ng of cell-free nucleic acid molecules. The method can comprise obtaining 1 femtogram (fg) to 200 ng.

Cell-free nucleic acids are nucleic acids not contained within or otherwise bound to a cell or in other words nucleic acids remaining in a sample after removing intact cells. Cell-free nucleic acids include DNA, RNA, and hybrids thereof, including genomic DNA, mitochondrial DNA, siRNA, miRNA, circulating RNA (cRNA), IRNA, rRNA, small nucleolar RNA (snoRNA), Piwi-interacting RNA (piRNA), long non-coding RNA (long ncRNA), or fragments of any of these. Cell-free nucleic acids can be double-stranded, single-stranded, or a hybrid thereof. A cell-free nucleic acid can be released into bodily fluid through secretion or cell death processes, e.g., cellular necrosis and apoptosis. Some cell-free nucleic acids are released into bodily fluid from cancer cells e.g., circulating tumor DNA, (ctDNA). Others are released from healthy cells. In some embodiments, cfDNA is cell-free fetal DNA (cffDNA) In some embodiments, cell free nucleic acids are produced by tumor cells. In some embodiments, cell free nucleic acids are produced by a mixture of tumor cells and non-tumor cells.

Cell-free nucleic acids have an exemplary size distribution of about 100-500 nucleotides, with molecules of 110 to about 230 nucleotides representing about 90% of molecules, with a mode of about 168 nucleotides and a second minor peak in a range between 240 to 440 nucleotides. Cell-free nucleic acids can be isolated from bodily fluids through a fractionation or partitioning step in which cell-free nucleic acids, as found in solution, are separated from intact cells and other non-soluble components of the bodily fluid. Partitioning may include techniques such as centrifugation or filtration. Alternatively, cells in bodily fluids can be lysed and cell-free and cellular nucleic acids processed together. Generally, after addition of buffers and wash steps, nucleic acids can be precipitated with alcohol. Further clean up steps may be used such as silica based columns to remove contaminants or salts. Non-specific bulk carrier nucleic acids, such as Cot-1 DNA, DNA or protein for bisulfite sequencing, hybridization, and/or ligation, may be added throughout the reaction to optimize certain aspects of the procedure such as yield.

After such processing, samples can include various forms of nucleic acid including double stranded DNA, single stranded DNA and single stranded RNA. In some embodiments, single stranded DNA and RNA can be converted to double stranded forms so they are included in subsequent processing and analysis steps.

### Analytes

Analytes can include nucleic acid analytes, and non-nucleic acid analytes. The disclosure provides for detecting genetic variations in biological samples from a subject. Biological samples may include polynucleotides from cancer cells. Polynucleotides may be DNA (e.g., genomic DNA, cDNA), RNA (e.g., mRNA, small RNAs), or any combination thereof. Biological samples may include tumor tissue, e.g., from a biopsy. In some cases, biological samples may include blood or saliva. In particular cases, biological samples may comprise cell free DNA (“cfDNA”) or circulating tumor DNA (“ctDNA”). Cell free DNA can be present in, e.g., blood.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquity lati on variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. This further includes receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

In general, the systems, apparatus, methods, and compositions can be used to analyze any number of analytes, further including both nucleic acid analytes and non-nucleic acid analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate. Methods for performing multiplexed assays to analyze two or more different analytes will be discussed in a subsequent section of this disclosure.

One or more nucleic acid analytes and/or non-nucleic acid analytes constitute a set of molecular interactions in a biological system under study (e.g., cells), which may be regarded as “interactome”—the molecular interactions that occur between molecules belonging to different biochemical families (proteins, nucleic acids, lipids, carbohydrates, etc.) and also within a given family. In various embodiments, an interactome is a protein-DNA interactome (network formed by transcription factors (and DNA or chromatin regulatory proteins) and their target genes. In other embodiments, interactome refers to protein-protein interaction network (PPI), or protein interaction network (PIN). The methods described herein allow for study and analysis of the interactome. Techniques such as proteogenomics (whole genome sequencing, whole exome sequencing and RNA-seq, and mass spectrometry as examples) can support study of the interactome.

### Analysis

The present methods can be used to diagnose presence of conditions, particularly cancer, in a subject, to characterize conditions (e.g., staging cancer or determining heterogeneity of a cancer), monitor response to treatment of a condition, effect prognosis risk of developing a condition or subsequent course of a condition. The present disclosure can also be useful in determining the efficacy of a particular treatment option. Successful treatment options may increase the amount of copy number variation or rare mutations detected in subject's blood if the treatment is successful as more cancers may die and shed DNA. In other examples, this may not occur. In another example, perhaps certain treatment options may be correlated with genetic profiles of cancers over time. This correlation may be useful in selecting a therapy. Additionally, if a cancer is observed to be in remission after treatment, the present methods can be used to monitor residual disease or recurrence of disease.

The types and number of cancers that may be detected may include blood cancers, brain cancers, lung cancers, skin cancers, nose cancers, throat cancers, liver cancers, bone cancers, lymphomas, pancreatic cancers, skin cancers, bowel cancers, rectal cancers, thyroid cancers, bladder cancers, kidney cancers, mouth cancers, stomach cancers, solid state tumors, heterogeneous tumors, homogenous tumors and the like. Type and/or stage of cancer can be detected from genetic variations including mutations, rare mutations, indels, copy number variations, transversions, translocations, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, and abnormal changes in nucleic acid 5-methylcytosine.

Genetic and other analyte data can also be used for characterizing a specific form of cancer. Cancers are often heterogeneous in both composition and staging. Genetic profile data may allow characterization of specific sub-types of cancer that may be important in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers can progress to become more aggressive and genetically unstable. Other cancers may remain benign, inactive or dormant. The system and methods of this disclosure may be useful in determining disease progression.

The present analyses are also useful in determining the efficacy of a particular treatment option. Successful treatment options may increase the amount of copy number variation or rare mutations detected in subject's blood if the treatment is successful as more cancers may die and shed DNA. In other examples, this may not occur. In another example, perhaps certain treatment options may be correlated with genetic profiles of cancers over time. This correlation may be useful in selecting a therapy. Additionally, if a cancer is observed to be in remission after treatment, the present methods can be used to monitor residual disease or recurrence of disease.

The present methods can also be used for detecting genetic variations in conditions other than cancer. Immune cells, such as B cells, may undergo rapid clonal expansion upon the presence of certain diseases. Clonal expansions may be monitored using copy number variation detection and certain immune states may be monitored. In this example, copy number variation analysis may be performed over time to produce a profile of how a particular disease may be progressing. Copy number variation or even rare mutation detection may be used to determine how a population of pathogens changes during the course of infection. This may be particularly important during chronic infections, such as HIV/AIDS or Hepatitis infections, whereby viruses may change life cycle state and/or mutate into more virulent forms during the course of infection. The present methods may be used to determine or profile rejection activities of the host body, as immune cells attempt to destroy transplanted tissue to monitor the status of transplanted tissue as well as altering the course of treatment or prevention of rejection.

Further, the methods of the disclosure may be used to characterize the heterogeneity of an abnormal condition in a subject. Such methods can include, e.g., generating a genetic profile of extracellular polynucleotides derived from the subject, wherein the genetic profile includes a plurality of data resulting from copy number variation and rare mutation analyses. In some embodiments, an abnormal condition is cancer. In some embodiments, the abnormal condition may be one resulting in a heterogeneous genomic population. In the example of cancer, some tumors are known to comprise tumor cells in different stages of the cancer. In other examples, heterogeneity may comprise multiple foci of disease. Again, in the example of cancer, there may be multiple tumor foci, perhaps where one or more foci are the result of metastases that have spread from a primary site.

The present methods can be used to generate or profile, fingerprint or set of data that is a summation of genetic information derived from different cells in a heterogeneous disease. This set of data may comprise copy number variation and mutation analyses alone or in combination.

The present methods can be used to diagnose, prognose, monitor or observe cancers. or other diseases. In some embodiments, the methods herein do not involve the diagnosing, prognosing or monitoring a fetus and as such are not directed to non-invasive prenatal testing. In other embodiments, these methodologies may be employed in a pregnant subject to diagnose, prognose, monitor or observe cancers or other diseases in an unborn subject whose DNA and other polynucleotides may co-circulate with maternal molecules.

### Determination of 5-Methylcytosine Pattern of Nucleic Acids

Bisulfite-based sequencing and variants thereof provides a means of determining the methylation pattern of a nucleic acid. In some embodiments, determining the methylation pattern includes distinguishing 5-methylcytosine (5mC) from non-methylated cytosine. In some embodiments, determining methylation pattern includes distinguishing N6-methyladenine from non-methylated adenine. In some embodiments, determining the methylation pattern includes distinguishing 5-hydroxymethylcytosine (5hmC), 5-formylcytosine (5fC), and 5-carboxylcytosine (5caC) from non-methylated cytosine. Examples of bisulfite sequencing include, but are not limited to oxidative bisulfite sequencing (OX-BS-seq), Tet-assisted bisulfite sequencing (TAB-seq), and reduced bisulfite sequencing (redBS-seq).

Oxidative bisulfite sequencing (OX-BS-seq) is used to distinguish between 5mC and 5hmC, by first converting the 5hmC to 5fC, and then proceeding with bisulfite sequencing as previously described. Tet-assisted bisulfite sequencing (TAB-seq) can also be used to distinguish 5mc and 5hmC. In TAB-seq, 5hmC is protected by glucosylation. A Tet enzyme is then used to convert 5mC to 5caC before proceeding with bisulfite sequencing, as previously described. Reduced bisulfite sequencing is used to distinguish 5fC from modified cytosines.

Generally, in bisulfite sequencing, a nucleic acid sample is divided into two aliquots and one aliquot is treated with bisulfite. The bisulfite converts native cytosine and certain modified cytosine nucleotides (e.g. 5-formylcytosine or 5-carboxylcytosine) to uracil whereas other modified cytosines (e.g., 5-methylcytosine, 5-hydroxymethylcytosine) are not converted. Comparison of nucleic acid sequences of molecules from the two aliquots indicates which cytosines were and were not converted to uracils. Consequently, cytosines which were and were not modified can be determined. The initial splitting of the sample into two aliquots is disadvantageous for samples containing only small amounts of nucleic acids, and/or composed of heterogeneous cell/tissue origins such as bodily fluids containing cell-free DNA.

The present disclosure provides methods allowing bisulfite sequencing and variants thereof. These methods work by linking nucleic acids in a population to a capture moiety, i.e., a label that can be captured or immobilized. Capture moieties include, without limitation, biotin, avidin, streptavidin, a nucleic acid including a particular nucleotide sequence, a hapten recognized by an antibody, and magnetically attractable particles. The extraction moiety can be a member of a binding pair, such as biotin/streptavidin or hapten/antibody. In some embodiments, a capture moiety that is attached to an analyte is captured by its binding pair which is attached to an isolatable moiety, such as a magnetically attractable particle or a large particle that can be sedimented through centrifugation. The capture moiety can be any type of molecule that allows affinity separation of nucleic acids bearing the capture moiety from nucleic acids lacking the capture moiety. Exemplary capture moieties are biotin which allows affinity separation by binding to streptavidin linked or linkable to a solid phase or an oligonucleotide, which allows affinity separation through binding to a complementary oligonucleotide linked or linkable to a solid phase. Following linking of capture moieties to sample nucleic acids, the sample nucleic acids serve as templates for amplification. Following amplification, the original templates remain linked to the capture moieties, but amplicons are not linked to capture moieties.

The capture moiety can be linked to sample nucleic acids as a component of an adapter, which may also provide amplification and/or sequencing primer binding sites. In some methods, sample nucleic acids are linked to adapters at both ends, with both adapters bearing a capture moiety. Preferably any cytosine residues in the adapters are modified, such as by 5methylcytosine, to protect against the action of bisulfite. In some instances, the capture moieties are linked to the original templates by a cleavable linkage (e.g., photocleavable desthiobiotin-TEG or uracil residues cleavable with USER™ enzyme, Chem. Commun. (Camb). 2015 Feb. 21; 51(15): 3266-3269), in which case the capture moieties can, if desired, be removed.

The amplicons are denatured and contacted with an affinity reagent for the capture tag. Original templates bind to the affinity reagent whereas nucleic acid molecules resulting from amplification do not. Thus, the original templates can be separated from nucleic acid molecules resulting from amplification.

Following separation or partition, the respective populations of nucleic acids (i.e., original templates and amplification products) can be subjected to bisulfite treatment with the original template population receiving bisulfite treatment and the amplification products not. Alternatively, the amplification products can be subjected to bisulfite treatment and the original template population is not. Following such treatment, the respective populations can be amplified (which in the case of the original template population converts uracils to thymines). The populations can also be subjected to biotin probe hybridization for enrichment. The respective populations are then analyzed and sequences compared to determine which cytosines were 5-methylated (or 5-hydroxylmethylated) in the original. Detection of a T nucleotide in the template population (corresponding to an unmethylated cytosine converted to uracil) and a C nucleotide at the corresponding position of the amplified population indicates an unmodified C. The presence of C's at corresponding positions of the original template and amplified populations indicates a modified C in the original sample.

In some embodiments, a method uses sequential DNA-seq and bisulfite-seq (BIS-seq) NGS library preparation of molecular tagged DNA libraries. This process is performed by labeling of adapters (e.g., biotin), DNA-seq amplification of whole library, parent molecule recovery (e.g. streptavidin bead pull down), bisulfite conversion and BIS-seq. In some embodiments, the method identifies 5-methylcytosine with single-base resolution, through sequential NGS-preparative amplification of parent library molecules with and without bisulfite treatment. This can be achieved by modifying the 5-methylated NGS-adapters (directional adapters; Y-shaped/forked with 5-methylcytosine replacing) used in BIS-seq with a label (e.g., biotin) on one of the two adapter strands. Sample DNA molecules are adapter ligated, and amplified (e.g., by PCR). As only the parent molecules will have a labeled adapter end, they can be selectively recovered from their amplified progeny by label-specific capture methods (e.g., streptavidin-magnetic beads). As the parent molecules retain 5-methylation marks, bisulfite conversion on the captured library will yield single-base resolution 5-methylation status upon BIS-seq, retaining molecular information to corresponding DNA-seq. In some embodiments, the bisulfite treated library can be combined with a non-treated library prior to enrichment/NGS by addition of a sample tag DNA sequence in standard multiplexed NGS workflow. As with BIS-seq workflows, bioinformatics analysis can be carried out for genomic alignment and 5-methylated base identification. In sum, this method provides the ability to selectively recover the parent, ligated molecules, carrying 5-methylcytosine marks, after library amplification, thereby allowing for parallel processing for bisulfite converted DNA. This overcomes the destructive nature of bisulfite treatment on the quality/sensitivity of the DNA-seq information extracted from a workflow. With this method, the recovered ligated, parent DNA molecules (via labeled adapters) allow amplification of the complete DNA library and parallel application of treatments that elicit epigenetic DNA modifications. The present disclosure discusses the use of BIS-seq methods to identify cytosine5-methylation (5-methylcytosine), but this is not limiting. Variants of BIS-seq have been developed to identify hydroxymethylated cytosines (5hmC; OX-BS-scq, TAB-scq), formylcytosine (5fC; redBS-seq) and carboxylcytosines. These methodologies can be implemented with the sequential/parallel library preparation described herein.

### Alternative Methods of Modified Nucleic Acid Analysis

The disclosure provides alternative methods for analyzing modified nucleic acids (e.g., methylated, linked to histones and other modifications discussed above). In some such methods, a population of nucleic acids bearing the modification to different extents (e.g., 0, 1, 2, 3, 4, 5 or more methyl groups per nucleic acid molecule) is contacted with adapters before fractionation of the population depending on the extent of the modification. Adapters attach to either one end or both ends of nucleic acid molecules in the population. Preferably, the adapters include different tags of sufficient numbers that the number of combinations of tags results in a low probability e.g., 95, 99 or 99.9% of two nucleic acids with the same start and stop points receiving the same combination of tags. Following attachment of adapters, the nucleic acids are amplified from primers binding to the primer binding sites within the adapters. Adapters, whether bearing the same or different tags, can include the same or different primer binding sites, but preferably adapters include the same primer binding site. Following amplification, the nucleic acids are contacted with an agent that preferably binds to nucleic acids bearing the modification (such as the previously described such agents). The nucleic acids are separated into at least two partitions differing in the extent to which the nucleic acids bear the modification from binding to the agents. For example, if the agent has affinity for nucleic acids bearing the modification, nucleic acids overrepresented in the modification (compared with median representation in the population) preferentially bind to the agent, whereas nucleic acids underrepresented for the modification do not bind or are more easily eluted from the agent. Following separation, the different partitions can then be subject to further processing steps, which typically include further amplification, and sequence analysis, in parallel but separately. Sequence data from the different partitions can then be compared.

Nucleic acids can be linked at both ends to Y-shaped adapters including primer binding sites and tags. The molecules are amplified. The amplified molecules are then fractionated by contact with an antibody preferentially binding to 5-methylcytosine to produce two partitions. One partition includes original molecules lacking methylation and amplification copies having lost methylation. The other partition includes original DNA molecules with methylation. The two partitions are then processed and sequenced separately with further amplification of the methylated partition. The sequence data of the two partitions can then be compared. In this example, tags are not used to distinguish between methylated and unmethylated DNA but rather to distinguish between different molecules within these partitions so that one can determine whether reads with the same start and stop points are based on the same or different molecules.

The disclosure provides further methods for analyzing a population of nucleic acid in which at least some of the nucleic acids include one or more modified cytosine residues, such as 5-methylcytosine and any of the other modifications described previously. In these methods, the population of nucleic acids is contacted with adapters including one or more cytosine residues modified at the 5C position, such as 5-methylcytosine. Preferably all cytosine residues in such adapters are also modified, or all such cytosines in a primer binding region of the adapters are modified. Adapters attach to both ends of nucleic acid molecules in the population. Preferably, the adapters include different tags of sufficient numbers that the number of combinations of tags results in a low probability e.g., 95, 99 or 99.9% of two nucleic acids with the same start and stop points receiving the same combination of tags. The primer binding sites in such adapters can be the same or different, but are preferably the same. After attachment of adapters, the nucleic acids are amplified from primers binding to the primer binding sites of the adapters. The amplified nucleic acids are split into first and second aliquots. The first aliquot is assayed for sequence data with or without further processing. The sequence data on molecules in the first aliquot is thus determined irrespective of the initial methylation state of the nucleic acid molecules. The nucleic acid molecules in the second aliquot are treated with bisulfite. This treatment converts unmodified cytosines to uracils. The bisulfite treated nucleic acids are then subjected to amplification primed by primers to the original primer binding sites of the adapters linked to nucleic acid. Only the nucleic acid molecules originally linked to adapters (as distinct from amplification products thereof) are now amplifiable because these nucleic acids retain cytosines in the primer binding sites of the adapters, whereas amplification products have lost the methylation of these cytosine residues, which have undergone conversion to uracils in the bisulfite treatment. Thus, only original molecules in the populations, at least some of which are methylated, undergo amplification. After amplification, these nucleic acids are subject to sequence analysis. Comparison of sequences determined from the first and second aliquots can indicate among other things, which cytosines in the nucleic acid population were subject to methylation.

Partitioning the Sample into a Plurality of Subsamples; Aspects of Samples; Analysis of Epigenetic Characteristics

In certain embodiments described herein, a population of different forms of nucleic acids (e.g., hypermethylated and hypomethylated DNA in a sample, such as a captured set of cfDNA as described herein) can be physically partitioned based on one or more characteristics of the nucleic acids prior to further analysis, e.g., differentially modifying or isolating a nucleobase, tagging, and/or sequencing. This approach can be used to determine, for example, whether certain sequences are hypermethylated or hypomethylated. In some embodiments, hypermethylation variable epigenetic target regions are analyzed to determine whether they show hypermethylation characteristic of tumor cells and/or hypomethylation variable epigenetic target regions are analyzed to determine whether they show hypomethylation characteristic of tumor cells. Additionally, by partitioning a heterogeneous nucleic acid population, one may increase rare signals, e.g., by enriching rare nucleic acid molecules that are more prevalent in one fraction (or partition) of the population. For example, a genetic variation present in hyper-methylated DNA but less (or not) in hypomethylated DNA can be more easily detected by partitioning a sample into hyper-methylated and hypo-methylated nucleic acid molecules. By analyzing multiple fractions of a sample, a multi-dimensional analysis of a single locus of a genome or species of nucleic acid can be performed and hence, greater sensitivity can be achieved.

In some instances, a heterogeneous nucleic acid sample is partitioned into two or more partitions (e.g., at least 3, 4, 5, 6 or 7 partitions). In some embodiments, each partition is differentially tagged. Tagged partitions can then be pooled together for collective sample prep and/or sequencing. The partitioning-tagging-pooling steps can occur more than once, with each round of partitioning occurring based on a different characteristics (examples provided herein) and tagged using differential tags that are distinguished from other partitions and partitioning means.

Examples of characteristics that can be used for partitioning include sequence length, methylation level, nucleosome binding, sequence mismatch, immunoprecipitation, and/or proteins that bind to DNA. Resulting partitions can include one or more of the following nucleic acid forms: single-stranded DNA (ssDNA), double-stranded DNA (dsDNA), shorter DNA fragments and longer DNA fragments. In some embodiments, partitioning based on a cytosine modification (e.g., cytosine methylation) or methylation generally is performed and is optionally combined with at least one additional partitioning step, which may be based on any of the foregoing characteristics or forms of DNA. In some embodiments, a heterogeneous population of nucleic acids is partitioned into nucleic acids with one or more epigenetic modifications and without the one or more epigenetic modifications. Examples of epigenetic modifications include presence or absence of methylation; level of methylation; type of methylation (e.g., 5-methylcytosine versus other types of methylation, such as adenine methylation and/or cytosine hydroxymethylation); and association and level of association with one or more proteins, such as histones. Alternatively or additionally, a heterogeneous population of nucleic acids can be partitioned into nucleic acid molecules associated with nucleosomes and nucleic acid molecules devoid of nucleosomes. Alternatively or additionally, a heterogeneous population of nucleic acids may be partitioned into single-stranded DNA (ssDNA) and double-stranded DNA (dsDNA). Alternatively, or additionally, a heterogeneous population of nucleic acids may be partitioned based on nucleic acid length (e.g., molecules of up to 160 bp and molecules having a length of greater than 160 bp).

In some instances, each partition (representative of a different nucleic acid form) is differentially labelled, and the partitions are pooled together prior to sequencing. In other instances, the different forms are separately sequenced. In some embodiments, a population of different nucleic acids is partitioned into two or more different partitions. Each partition is representative of a different nucleic acid form, and a first partition (also referred to as a subsample) includes DNA with a cytosine modification in a greater proportion than a second subsample. Each partition is distinctly tagged. The first subsample is subjected to a procedure that affects a first nucleobase in the DNA differently from a second nucleobase in the DNA of the first subsample, wherein the first nucleobase is a modified or unmodified nucleobase, the second nucleobase is a modified or unmodified nucleobase different from the first nucleobase, and the first nucleobase and the second nucleobase have the same base pairing specificity. The tagged nucleic acids are pooled together prior to sequencing. Sequence reads are obtained and analyzed, including to distinguish the first nucleobase from the second nucleobase in the DNA of the first subsample, in silico. Tags are used to sort reads from different partitions. Analysis to detect genetic variants can be performed on a partition-by-partition level, as well as whole nucleic acid population level. For example, analysis can include in silico analysis to determine genetic variants, such as CNV, SNV, indel, fusion in nucleic acids in each partition. In some instances, in silico analysis can include determining chromatin structure. For example, coverage of sequence reads can be used to determine nucleosome positioning in chromatin. Higher coverage can correlate with higher nucleosome occupancy in genomic region while lower coverage can correlate with lower nucleosome occupancy or nucleosome depleted region (NDR).

Samples can include nucleic acids varying in modifications including post-replication modifications to nucleotides and binding, usually noncovalently, to one or more proteins.

In an embodiment, the population of nucleic acids is one obtained from a serum, plasma or blood sample from a subject suspected of having neoplasia, a tumor, or cancer or previously diagnosed with neoplasia, a tumor, or cancer. The population of nucleic acids includes nucleic acids having varying levels of methylation. Methylation can occur from any one or more post-replication or transcriptional modifications. Post-replication modifications include modifications of the nucleotide cytosine, particularly at the 5-position of the nucleobase, e.g., 5-methylcytosine, 5-hydroxymethylcytosine, 5-formylcytosine and 5-carboxylcytosine. The affinity agents can be antibodies with the desired specificity, natural binding partners or variants thereof (Bock et al., Nat Biotech 28:1106-1114 (2010); Song et al., Nat Biotech 29:68-72 (2011)), or artificial peptides selected e.g., by phage display to have specificity to a given target.

Examples of capture moieties contemplated herein include methyl binding domain (MBDs) and methyl binding proteins (MBPs) as described herein, including proteins such as MeCP2 and antibodies preferentially binding to 5-methylcytosine. Likewise, partitioning of different forms of nucleic acids can be performed using histone binding proteins which can separate nucleic acids bound to histones from free or unbound nucleic acids. Examples of histone binding proteins that can be used in the methods disclosed herein include RBBP4, RbAp48 and SANT domain peptides. Although for some affinity agents and modifications, binding to the agent may occur in an essentially all or none manner depending on whether a nucleic acid bears a modification, the separation may be one of degree. In such instances, nucleic acids overrepresented in a modification bind to the agent at a greater extent that nucleic acids underrepresented in the modification. Alternatively, nucleic acids having modifications may bind in an all or nothing manner. But then, various levels of modifications may be sequentially eluted from the binding agent.

For example, in some embodiments, partitioning can be binary or based on degree/level of modifications. For example, all methylated fragments can be partitioned from unmethylated fragments using methyl-binding domain proteins (e.g., MethylMiner Methylated DNA Enrichment Kit (ThermoFisher Scientific)). Subsequently, additional partitioning may involve eluting fragments having different levels of methylation by adjusting the salt concentration in a solution with the methyl-binding domain and bound fragments. As salt concentration increases, fragments having greater methylation levels are eluted. In some instances, the final partitions are representative of nucleic acids having different extents of modifications (overrepresentative or underrepresentative of modifications). Overrepresentation and underrepresentation can be defined by the number of modifications born by a nucleic acid relative to the median number of modifications per strand in a population. For example, if the median number of 5-methylcytosine residues in nucleic acid in a sample is 2, a nucleic acid including more than two 5-methylcytosine residues is overrepresented in this modification and a nucleic acid with 1 or zero 5-methylcytosine residues is underrepresented. The effect of the affinity separation is to enrich for nucleic acids overrepresented in a modification in a bound phase and for nucleic acids underrepresented in a modification in an unbound phase (i.e. in solution). The nucleic acids in the bound phase can be eluted before subsequent processing.

When using MethylMiner Methylated DNA Enrichment Kit (ThermoFisher Scientific) various levels of methylation can be partitioned using sequential elutions. For example, a hypomethylated partition (e.g., no methylation) can be separated from a methylated partition by contacting the nucleic acid population with the MBD from the kit, which is attached to magnetic beads. The beads are used to separate out the methylated nucleic acids from the non-methylated nucleic acids. Subsequently, one or more elution steps are performed sequentially to elute nucleic acids having different levels of methylation. For example, a first set of methylated nucleic acids can be eluted at a salt concentration of 160 mM or higher, e.g., at least 150 mM, at least 200 mM, at least 300 mM, at least 400 mM, at least 500 mM, at least 600 mM, at least 700 mM, at least 800 mM, at least 900 mM, at least 1000 mM, or at least 2000 mM. After such methylated nucleic acids are eluted, magnetic separation is once again used to separate higher levels of methylated nucleic acids from those with lower level of methylation. The elution and magnetic separation steps can repeat themselves to create various partitions such as a hypomethylated partition (representative of no methylation), a methylated partition (representative of low level of methylation), and a hyper methylated partition (representative of high level of methylation).

In some methods, nucleic acids bound to an agent used for affinity separation are subjected to a wash step. The wash step washes off nucleic acids weakly bound to the affinity agent. Such nucleic acids can be enriched in nucleic acids having the modification to an extent close to the mean or median (i.e., intermediate between nucleic acids remaining bound to the solid phase and nucleic acids not binding to the solid phase on initial contacting of the sample with the agent). The affinity separation results in at least two, and sometimes three or more partitions of nucleic acids with different extents of a modification. While the partitions are still separate, the nucleic acids of at least one partition, and usually two or three (or more) partitions are linked to nucleic acid tags, usually provided as components of adapters, with the nucleic acids in different partitions receiving different tags that distinguish members of one partition from another. The tags linked to nucleic acid molecules of the same partition can be the same or different from one another. But if different from one another, the tags may have part of their code in common so as to identify the molecules to which they are attached as being of a particular partition. For further details regarding portioning nucleic acid samples based on characteristics such as methylation, see WO2018/119452, which is incorporated herein by reference. In some embodiments, the nucleic acid molecules can be fractionated into different partitions based on the nucleic acid molecules that are bound to a specific protein or a fragment thereof and those that are not bound to that specific protein or fragment thereof.

Nucleic acid molecules can be fractionated based on DNA-protein binding. Protein-DNA complexes can be fractionated based on a specific property of a protein. Examples of such properties include various epitopes, modifications (e.g., histone methylation or acetylation) or enzymatic activity. Examples of proteins which may bind to DNA and serve as a basis for fractionation may include, but are not limited to, protein A and protein G. Any suitable method can be used to fractionate the nucleic acid molecules based on protein bound regions. Examples of methods used to fractionate nucleic acid molecules based on protein bound regions include, but are not limited to, SDS-PAGE, chromatin-immuno-precipitation (ChIP), heparin chromatography, and asymmetrical field flow fractionation (AF4).

In some embodiments, partitioning of the nucleic acids is performed by contacting the nucleic acids with a methylation binding domain (“MBD”) of a methylation binding protein (“MBP”). MBD binds to 5-methylcytosine (5mC). MBD is coupled to paramagnetic beads, such as Dynabeads® M-280 Streptavidin via a biotin linker. Partitioning into fractions with different extents of methylation can be performed by eluting fractions by increasing the NaCl concentration.

An exemplary method for molecular tag identification of MBD-bead partitioned libraries through NGS is as follows:

Physical partitioning of an extracted DNA sample (e.g., extracted blood plasma DNA from a human sample) using a methyl-binding domain protein-bead purification kit, saving all elutions from process for downstream processing.

Parallel application of differential molecular tags and NGS-enabling adapter sequences to each partition. For example, the hypermethylated, residual methylation (‘wash’), and hypomethylated partitions are ligated with NGS-adapters with molecular tags.

Re-combining all molecular tagged partitions, and subsequent amplification using adapter-specific DNA primer sequences.

Enrichment/hybridization of re-combined and amplified total library, targeting genomic regions of interest (e.g., cancer-specific genetic variants and differentially methylated regions).

Re-amplification of the enriched total DNA library, appending a sample tag. Different samples are pooled and assayed in multiplex on an NGS instrument.

Bioinformatics analysis of NGS data, with the molecular tags being used to identify unique molecules, as well deconvolution of the sample into molecules that were differentially MBD-partitioned. This analysis can yield information on relative 5-methylcytosine for genomic regions, concurrent with standard genetic sequencing/variant detection.

Examples of MBPs contemplated herein include, but are not limited to:
(a) MeCP2 is a protein preferentially binding to 5-methyl-cytosine over unmodified cytosine.
(b) RPL26, PRP8 and the DNA mismatch repair protein MHS6 preferentially bind to 5-hydroxymethyl-cytosine over unmodified cytosine.
(c) FOXK1, FOXK2, FOXP1, FOXP4 and FOXI3 preferably bind to 5-formyl-cytosine over unmodified cytosine (Iurlaro et al., Genome Biol. 14: R119 (2013)).
(d) Antibodies specific to one or more methylated nucleotide bases.

In general, elution is a function of number of methylated sites per molecule, with molecules having more methylation eluting under increased salt concentrations. To elute the DNA into distinct populations based on the extent of methylation, one can use a series of elution buffers of increasing NaCl concentration. Salt concentration can range from about 100 nM to about 2500 mM NaCl. In one embodiment, the process results in three (3) partitions. Molecules are contacted with a solution at a first salt concentration and including a molecule including a methyl binding domain, which molecule can be attached to a capture moiety, such as streptavidin. At the first salt concentration a population of molecules will bind to the MBD and a population will remain unbound. The unbound population can be separated as a “hypomethylated” population. For example, a first partition representative of the hypomethylated form of DNA is that which remains unbound at a low salt concentration, e.g., 100 mM or 160 mM. A second partition representative of intermediate methylated DNA is eluted using an intermediate salt concentration, e.g., between 100 mM and 2000 mM concentration. This is also separated from the sample. A third partition representative of hypermethylated form of DNA is eluted using a high salt concentration, e.g., at least about 2000 mM.

The disclosure provides further methods for analyzing a population of nucleic acids in which at least some of the nucleic acids include one or more modified cytosine residues, such as 5-methylcytosine and any of the other modifications described previously. In these methods, after partitioning, the subsamples of nucleic acids are contacted with adapters including one or more cytosine residues modified at the 5C position, such as 5-methylcytosine. Preferably all cytosine residues in such adapters are also modified, or all such cytosines in a primer binding region of the adapters are modified. Adapters attach to both ends of nucleic acid molecules in the population. Preferably, the adapters include different tags of sufficient numbers that the number of combinations of tags results in a low probability e.g., 95, 99 or 99.9% of two nucleic acids with the same start and stop points receiving the same combination of tags. The primer binding sites in such adapters can be the same or different, but are preferably the same. After attachment of adapters, the nucleic acids are amplified from primers binding to the primer binding sites of the adapters. The amplified nucleic acids are split into first and second aliquots. The first aliquot is assayed for sequence data with or without further processing. The sequence data on molecules in the first aliquot is thus determined irrespective of the initial methylation state of the nucleic acid molecules. The nucleic acid molecules in the second aliquot are subjected to a procedure that affects a first nucleobase in the DNA differently from a second nucleobase in the DNA, wherein the first nucleobase includes a cytosine modified at the 5 position, and the second nucleobase includes unmodified cytosine. This procedure may be bisulfite treatment or another procedure that converts unmodified cytosines to uracils. The nucleic acids subjected to the procedure are then amplified with primers to the original primer binding sites of the adapters linked to nucleic acid. Only the nucleic acid molecules originally linked to adapters (as distinct from amplification products thereof) are now amplifiable because these nucleic acids retain cytosines in the primer binding sites of the adapters, whereas amplification products have lost the methylation of these cytosine residues, which have undergone conversion to uracils in the bisulfite treatment. Thus, only original molecules in the populations, at least some of which are methylated, undergo amplification. After amplification, these nucleic acids are subject to sequence analysis. Comparison of sequences determined from the first and second aliquots can indicate among other things, which cytosines in the nucleic acid population were subject to methylation.

Such an analysis can be performed using the following exemplary procedure. After partitioning, methylated DNA is linked to Y-shaped adapters at both ends including primer binding sites and tags. The cytosines in the adapters are modified at the 5 position (e.g., 5-methylated). The modification of the adapters serves to protect the primer binding sites in a subsequent conversion step (e.g., bisulfite treatment, TAP conversion, or any other conversion that does not affect the modified cytosine but affects unmodified cytosine). After attachment of adapters, the DNA molecules are amplified. The amplification product is split into two aliquots for sequencing with and without conversion. The aliquot not subjected to conversion can be subjected to sequence analysis with or without further processing. The other aliquot is subjected to a procedure that affects a first nucleobase in the DNA differently from a second nucleobase in the DNA, wherein the first nucleobase includes a cytosine modified at the 5 position, and the second nucleobase includes unmodified cytosine. This procedure may be bisulfite treatment or another procedure that converts unmodified cytosines to uracils. Only primer binding sites protected by modification of cytosines can support amplification when contacted with primers specific for original primer binding sites. Thus, only original molecules and not copies from the first amplification are subjected to further amplification. The further amplified molecules are then subjected to sequence analysis. Sequences can then be compared from the two aliquots. As in the separation scheme discussed above, nucleic acid tags in adapters are not used to distinguish between methylated and unmethylated DNA but to distinguish nucleic acid molecules within the same partition.

Subjecting the First Subsample to a Procedure that Affects a First Nucleobase in the DNA Differently from a Second Nucleobase in the DNA of the First Subsample

Methods disclosed herein comprise a step of subjecting the first subsample to a procedure that affects a first nucleobase in the DNA differently from a second nucleobase in the DNA of the first subsample, wherein the first nucleobase is a modified or unmodified nucleobase, the second nucleobase is a modified or unmodified nucleobase different from the first nucleobase, and the first nucleobase and the second nucleobase have the same base pairing specificity. In some embodiments, if the first nucleobase is a modified or unmodified adenine, then the second nucleobase is a modified or unmodified adenine; if the first nucleobase is a modified or unmodified cytosine, then the second nucleobase is a modified or unmodified cytosine; if the first nucleobase is a modified or unmodified guanine, then the second nucleobase is a modified or unmodified guanine; and if the first nucleobase is a modified or unmodified thymine, then the second nucleobase is a modified or unmodified thymine (where modified and unmodified uracil are encompassed within modified thymine for the purpose of this step).

In some embodiments, the first nucleobase is a modified or unmodified cytosine, then the second nucleobase is a modified or unmodified cytosine. For example, first nucleobase may comprise unmodified cytosine (C) and the second nucleobase may comprise one or more of 5-methylcytosine (mC) and 5-hydroxymethylcytosine (hmC). Alternatively, the second nucleobase may comprise C and the first nucleobase may comprise one or more of mC and hmC. Other combinations are also possible, as indicated, e.g., in the Summary above and the following discussion, such as where one of the first and second nucleobases includes mC and the other includes hmC.

In some embodiments, the procedure that affects a first nucleobase in the DNA differently from a second nucleobase in the DNA of the first subsample includes bisulfite conversion. Treatment with bisulfite converts unmodified cytosine and certain modified cytosine nucleotides (e.g. 5-formyl cytosine (fC) or 5-carboxylcytosine (caC)) to uracil whereas other modified cytosines (e.g., 5-methylcytosine, 5-hydroxylmethylcystosine) are not converted. Thus, where bisulfite conversion is used, the first nucleobase includes one or more of unmodified cytosine, 5-formyl cytosine, 5-carboxylcytosine, or other cytosine forms affected by bisulfite, and the second nucleobase may comprise one or more of mC and hmC, such as mC and optionally hmC. Sequencing of bisulfite-treated DNA identifies positions that are read as cytosine as being mC or hmC positions. Meanwhile, positions that are read as T are identified as being T or a bisulfite-susceptible form of C, such as unmodified cytosine, 5-formyl cytosine, or 5-carboxylcytosine. Performing bisulfite conversion on a first subsample as described herein thus facilitates identifying positions containing mC or hmC using the sequence reads obtained from the first subsample. For an exemplary description of bisulfite conversion, see, e.g., Moss et al., Nat Commun. 2018; 9:5068.

In some embodiments, the procedure that affects a first nucleobase in the DNA differently from a second nucleobase in the DNA of the first subsample includes oxidative bisulfite (Ox-BS) conversion. In some embodiments, the procedure that affects a first nucleobase in the DNA differently from a second nucleobase in the DNA of the first subsample includes Tet-assisted bisulfite (TAB) conversion. In some embodiments, the procedure that affects a first nucleobase in the DNA differently from a second nucleobase in the DNA of the first subsample includes Tet-assisted conversion with a substituted borane reducing agent, optionally wherein the substituted borane reducing agent is 2-picoline borane, borane pyridine, tert-butylamine borane, or ammonia borane. In some embodiments, the procedure that affects a first nucleobase in the DNA differently from a second nucleobase in the DNA of the first subsample includes chemical-assisted conversion with a substituted borane reducing agent, optionally wherein the substituted borane reducing agent is 2-picoline borane, borane pyridine, tert-butylamine borane, or ammonia borane. In some embodiments, the procedure that affects a first nucleobase in the DNA differently from a second nucleobase in the DNA of the first subsample includes APOBEC-coupled epigenetic (ACE) conversion.

In some embodiments, procedure that affects a first nucleobase in the DNA differently from a second nucleobase in the DNA of the first subsample includes enzymatic conversion of the first nucleobase, e.g., as in EM-Seq. Sec, e.g., Vaisvila R, et al. (2019) EM-seq: Detection of DNA methylation at single base resolution from picograms of DNA. bioRxiv; DOI: 10.1101/2019.12.20.884692, available at www.biorxiv.org/content/10.1101/2019.12.20.884692v1. For example, TET2 and T4-BGT can be used to convert 5mC and 5hmC into substrates that cannot be deaminated by a deaminase (e.g., APOBEC3A), and then a deaminase (e.g., APOBEC3A) can be used to deaminate unmodified cytosines converting them to uracils.

In some embodiments, the procedure that affects a first nucleobase in the DNA differently from a second nucleobase in the DNA of the first subsample includes separating DNA originally including the first nucleobase from DNA not originally including the first nucleobase.

In some embodiments, the first nucleobase is a modified or unmodified adenine, and the second nucleobase is a modified or unmodified adenine. In some embodiments, the modified adenine is N6-methyladenine (mA). In some embodiments, the modified adenine is one or more of N6-methyladenine (mA), N6-hydroxymethyladenine (hmA), or N6-formyladenine (fA).

Techniques including methylated DNA immunoprecipitation (MeDIP) can be used to separate DNA containing modified bases such as mA from other DNA. See, e.g., Kumar et al., Frontiers Genet. 2018; 9:640; Greer et al., Cell 2015; 161:868-878. An antibody specific for mA is described in Sun et al., Bioessays 2015; 37:1155-62. Antibodies for various modified nucleobases, such as forms of thymine/uracil including halogenated forms such as 5-bromouracil, are commercially available. Various modified bases can also be detected based on alterations in their base-pairing specificity. For example, hypoxanthine is a modified form of adenine that can result from deamination and is read in sequencing as a G. See, e.g., U.S. Pat. No. 8,486,630; Brown, Genomes, 2nd Ed., John Wiley & Sons, Inc., New York, N.Y., 2002, chapter 14, “Mutation, Repair, and Recombination.”

### Enriching/Capturing Step, Amplification, Adaptors, Barcodes

In some embodiments, methods disclosed herein comprise a step of capturing one or more sets of target regions of DNA, such as cfDNA. Capture may be performed using any suitable approach known in the art. In some embodiments, capturing includes contacting the DNA to be captured with a set of target-specific probes. The set of target-specific probes may have any of the features described herein for sets of target-specific probes, including but not limited to in the embodiments set forth above and the sections relating to probes below. Capturing may be performed on one or more subsamples prepared during methods disclosed herein. In some embodiments, DNA is captured from at least the first subsample or the second subsample, e.g., at least the first subsample and the second subsample. Where the first subsample undergoes a separation step (e.g., separating DNA originally including the first nucleobase (e.g., hmC) from DNA not originally including the first nucleobase, such as hmC-seal), capturing may be performed on any, any two, or all of the DNA originally including the first nucleobase (e.g., hmC), the DNA not originally including the first nucleobase, and the second subsample. In some embodiments, the subsamples are differentially tagged (e.g., as described herein) and then pooled before undergoing capture.

The capturing step may be performed using conditions suitable for specific nucleic acid hybridization, which generally depend to some extent on features of the probes such as length, base composition, etc. Those skilled in the art will be familiar with appropriate conditions given general knowledge in the art regarding nucleic acid hybridization. In some embodiments, complexes of target-specific probes and DNA are formed.

In some embodiments, a method described herein includes capturing cfDNA obtained from a test subject for a plurality of sets of target regions. The target regions comprise epigenetic target regions, which may show differences in methylation levels and/or fragmentation patterns depending on whether they originated from a tumor or from healthy cells. The target regions also comprise sequence-variable target regions, which may show differences in sequence depending on whether they originated from a tumor or from healthy cells. The capturing step produces a captured set of cfDNA molecules, and the cfDNA molecules corresponding to the sequence-variable target region set are captured at a greater capture yield in the captured set of cfDNA molecules than cfDNA molecules corresponding to the epigenetic target region set. For additional discussion of capturing steps, capture yields, and related aspects, see WO2020/160414, which is incorporated herein by reference for all purposes.

In some embodiments, a method described herein includes contacting cfDNA obtained from a test subject with a set of target-specific probes, wherein the set of target-specific probes is configured to capture cfDNA corresponding to the sequence-variable target region set at a greater capture yield than cfDNA corresponding to the epigenetic target region set.

It can be beneficial to capture cfDNA corresponding to the sequence-variable target region set at a greater capture yield than cfDNA corresponding to the epigenetic target region set because a greater depth of sequencing may be necessary to analyze the sequence-variable target regions with sufficient confidence or accuracy than may be necessary to analyze the epigenetic target regions. The volume of data needed to determine fragmentation patterns (e.g., to test fsor perturbation of transcription start sites or CTCF binding sites) or fragment abundance (e.g., in hypermethylated and hypomethylated partitions) is generally less than the volume of data needed to determine the presence or absence of cancer-related sequence mutations. Capturing the target region sets at different yields can facilitate sequencing the target regions to different depths of sequencing in the same sequencing run (e.g., using a pooled mixture and/or in the same sequencing cell).

In various embodiments, the methods further comprise sequencing the captured cfDNA, e.g., to different degrees of sequencing depth for the epigenetic and sequence-variable target region sets, consistent with the discussion herein. In some embodiments, complexes of target-specific probes and DNA are separated from DNA not bound to target-specific probes. For example, where target-specific probes are bound covalently or noncovalently to a solid support, a washing or aspiration step can be used to separate unbound material. Alternatively, where the complexes have chromatographic properties distinct from unbound material (e.g., where the probes comprise a ligand that binds a chromatographic resin), chromatography can be used.

As discussed in detail elsewhere herein, the set of target-specific probes may comprise a plurality of sets such as probes for a sequence-variable target region set and probes for an epigenetic target region set. In some such embodiments, the capturing step is performed with the probes for the sequence-variable target region set and the probes for the epigenetic target region set in the same vessel at the same time, e.g., the probes for the sequence-variable and epigenetic target region sets are in the same composition. This approach provides a relatively streamlined workflow. In some embodiments, the concentration of the probes for the sequence-variable target region set is greater that the concentration of the probes for the epigenetic target region set.

Alternatively, the capturing step is performed with the sequence-variable target region probe set in a first vessel and with the epigenetic target region probe set in a second vessel, or the contacting step is performed with the sequence-variable target region probe set at a first time and a first vessel and the epigenetic target region probe set at a second time before or after the first time. This approach allows for preparation of separate first and second compositions including captured DNA corresponding to the sequence-variable target region set and captured DNA corresponding to the epigenetic target region set. The compositions can be processed separately as desired (e.g., to fractionate based on methylation as described elsewhere herein) and recombined in appropriate proportions to provide material for further processing and analysis such as sequencing.

In some embodiments, the DNA is amplified. In some embodiments, amplification is performed before the capturing step. In some embodiments, amplification is performed after the capturing step.

In some embodiments, adapters are included in the DNA. This may be done concurrently with an amplification procedure, e.g., by providing the adapters in a 5′ portion of a primer, e.g., as described above. Alternatively, adapters can be added by other approaches, such as ligation.

In some embodiments, tags, which may be or include barcodes, are included in the DNA. Tags can facilitate identification of the origin of a nucleic acid. For example, barcodes can be used to allow the origin (e.g., subject) whence the DNA came to be identified following pooling of a plurality of samples for parallel sequencing. This may be done concurrently with an amplification procedure, e.g., by providing the barcodes in a 5′ portion of a primer, e.g., as described above. In some embodiments, adapters and tags/barcodes are provided by the same primer or primer set. For example, the barcode may be located 3′ of the adapter and 5′ of the target-hybridizing portion of the primer. Alternatively, barcodes can be added by other approaches, such as ligation, optionally together with adapters in the same ligation substrate.

Additional details regarding amplification, tags, and barcodes are discussed in the “General Features of the Methods” section below, which can be combined to the extent practicable with any of the foregoing embodiments and the embodiments set forth in the introduction and summary section.

### Captured Set

In some embodiments, a captured set of DNA (e.g., cfDNA) is provided. With respect to the disclosed methods, the captured set of DNA may be provided, e.g., by performing a capturing step after a partitioning step as described herein. The captured set may comprise DNA corresponding to a sequence-variable target region set, an epigenetic target region set, or a combination thereof. In some embodiments the quantity of captured sequence-variable target region DNA is greater than the quantity of the captured epigenetic target region DNA, when normalized for the difference in the size of the targeted regions (footprint size).

Alternatively, first and second captured sets may be provided, including, respectively, DNA corresponding to a sequence-variable target region set and DNA corresponding to an epigenetic target region set. The first and second captured sets may be combined to provide a combined captured set.

In some embodiments in which a captured set including DNA corresponding to the sequence-variable target region set and the epigenetic target region set includes a combined captured set as discussed above, the DNA corresponding to the sequence-variable target region set may be present at a greater concentration than the DNA corresponding to the epigenetic target region set, e.g., a 1.1 to 1.2-fold greater concentration, a 1.2- to 1.4-fold greater concentration, a 1.4- to 1.6-fold greater concentration, a 1.6- to 1.8-fold greater concentration, a 1.8- to 2.0-fold greater concentration, a 2.0- to 2.2-fold greater concentration, a 2.2- to 2.4-fold greater concentration a 2.4- to 2.6-fold greater concentration, a 2.6- to 2.8-fold greater concentration, a 2.8- to 3.0-fold greater concentration, a 3.0- to 3.5-fold greater concentration, a 3.5- to 4.0, a 4.0- to 4.5-fold greater concentration, a 4.5- to 5.0-fold greater concentration, a 5.0- to 5.5-fold greater concentration, a 5.5- to 6.0-fold greater concentration, a 6.0- to 6.5-fold greater concentration, a 6.5- to 7.0-fold greater, a 7.0- to 7.5-fold greater concentration, a 7.5- to 8.0-fold greater concentration, an 8.0- to 8.5-fold greater concentration, an 8.5- to 9.0-fold greater concentration, a 9.0- to 9.5-fold greater concentration, 9.5- to 10.0-fold greater concentration, a 10- to 11-fold greater concentration, an 11- to 12-fold greater concentration a 12- to 13-fold greater concentration, a 13- to 14-fold greater concentration, a 14- to 15-fold greater concentration, a 15- to 16-fold greater concentration, a 16- to 17-fold greater concentration, a 17- to 18-fold greater concentration, an 18- to 19-fold greater concentration, a 19- to 20-fold greater concentration, a 20- to 30-fold greater concentration, a 30- to 40-fold greater concentration, a 40- to 50-fold greater concentration, a 50- to 60-fold greater concentration, a 60- to 70-fold greater concentration, a 70- to 80-fold greater concentration, a 80- to 90-fold greater concentration, a 90- to 100-fold greater concentration, a 10- to 20-fold greater concentration, a 10- to 40-fold greater concentration, a 10- to 50-fold greater concentration, a 10- to 70-fold greater concentration, or a 10- to 100-fold greater concentration. The degree of difference in concentrations accounts for normalization for the footprint sizes of the target regions, as discussed in the definition section.

### Epigenetic Target Region Set

The epigenetic target region set may comprise one or more types of target regions likely to differentiate DNA from neoplastic (e.g., tumor or cancer) cells and from healthy cells, e.g., non-neoplastic circulating cells. Exemplary types of such regions are discussed in detail herein. The epigenetic target region set may also comprise one or more control regions, e.g., as described herein. In some embodiments, the epigenetic target region set has a footprint of at least 100 kb, e.g., at least 200 kb, at least 300 kb, or at least 400 kb. In some embodiments, the epigenetic target region set has a footprint in the range of 100-1000 kb, e.g., 100-200 kb, 200-300 kb, 300-400 kb, 400-500 kb, 500-600 kb, 600-700 kb, 700-800 kb, 800-900 kb, and 900-1,000 kb.

### Hypermethylation Variable Target Regions

In some embodiments, the epigenetic target region set includes one or more hypermethylation variable target regions. In general, hypermethylation variable target regions refer to regions where an increase in the level of observed methylation, e.g., in a cfDNA sample, indicates an increased likelihood that a sample (e.g., of cfDNA) contains DNA produced by neoplastic cells, such as tumor or cancer cells. For example, hypermethylation of promoters of tumor suppressor genes has been observed repeatedly. See, e.g., Kang et al., Genome Biol. 18:53 (2017) and references cited therein. In an example, hypermethylation variable target regions can include regions that do not necessarily differ in methylation in cancerous tissue relative to DNA from healthy tissue of the same type, but do differ in methylation (e.g., have more methylation) relative to cfDNA that is typical in healthy subjects. Where, for example, the presence of a cancer results in increased cell death such as apoptosis of cells of the tissue type corresponding to the cancer, such a cancer can be detected at least in part using such hypermethylation variable target regions. In some embodiments, hypermethylation variable target regions include one or more genomic regions, where the cfDNA molecules in those regions do not differ in methylation state in cancer subjects relative to cfDNA from healthy subjects, but the presence/increased quantity of hypermethylated cfDNA in those regions is indicative of a particular tissue type (e.g., cancer origin) and is presented as cfDNA with increased apoptosis (e.g. tumor shedding) into circulation.

Hypermethylation target regions may be obtained, e.g., from the Cancer Genome Atlas. Kang et al., Genome Biology 18:53 (2017), describe construction of a probabilistic method called CancerLocator using hypermethylation target regions from breast, colon, kidney, liver, and lung. In some embodiments, the hypermethylation target regions can be specific to one or more types of cancer. Accordingly, in some embodiments, the hypermethylation target regions include one, two, three, four, or five subsets of hypermethylation target regions that collectively show hypermethylation in one, two, three, four, or five of breast, colon, kidney, liver, and lung cancers.

In some embodiments, the probes for the epigenetic target region set comprise probes specific for one or more hypermethylation variable target regions. The hypermethylation variable target regions may be any of those set forth above. For example, in some embodiments, the probes specific for hypermethylation variable target regions comprise probes specific for a plurality of loci listed in Table 1, e.g., at least 10%, 20%, 30%, 40%, 50%, 60%, 70%, 80%, 90%, or 100% of the loci listed in Table 1. In some embodiments, the probes specific for hypermethylation variable target regions comprise probes specific for a plurality of loci listed in Table 2, e.g., at least 10%, 20%, 30%, 40%, 50%, 60%, 70%, 80%, 90%, or 100% of the loci listed in Table 2. In some embodiments, the probes specific for hypermethylation variable target regions comprise probes specific for a plurality of loci listed in Table 1 or Table 2, e.g., at least 10%, 20%, 30%, 40%, 50%, 60%, 70%, 80%, 90%, or 100% of the loci listed in Table 1 or Table 2. In some embodiments, for each locus included as a target region, there may be one or more probes with a hybridization site that binds between the transcription start site and the stop codon (the last stop codon for genes that are alternatively spliced) of the gene. In some embodiments, the one or more probes bind within 300 bp of the listed position, e.g., within 200 or 100 bp. In some embodiments, a probe has a hybridization site overlapping the position listed above. In some embodiments, the probes specific for the hypermethylation target regions include probes specific for one, two, three, four, or five subsets of hypermethylation target regions that collectively show hypermethylation in one, two, three, four, or five of breast, colon, kidney, liver, and lung cancers.

### Hypomethylation Variable Target Regions

Global hypomethylation is a commonly observed phenomenon in various cancers. Sec, e.g., Hon et al., Genome Res. 22:246-258 (2012) (breast cancer); Ehrlich, Epigenomics 1:239-259 (2009) (review article noting observations of hypomethylation in colon, ovarian, prostate, leukemia, hepatocellular, and cervical cancers). For example, regions such as repeated elements, e.g., LINE1 elements, Alu elements, centromeric tandem repeats, pericentromeric tandem repeats, and satellite DNA, and intergenic regions that are ordinarily methylated in healthy cells may show reduced methylation in tumor cells. Accordingly, in some embodiments, the epigenetic target region set includes hypomethylation variable target regions, where a decrease in the level of observed methylation indicates an increased likelihood that a sample (e.g., of cfDNA) contains DNA produced by neoplastic cells, such as tumor or cancer cells. In an example, hypomethylation variable target regions can include regions that do not necessarily differ in methylation state in cancerous tissue relative to DNA from healthy tissue of the same type, but do differ in methylation (e.g., are less methylated) relative to cfDNA that is typical in healthy subjects. Where, for example, the presence of a cancer results in increased cell death such as apoptosis of cells of the tissue type corresponding to the cancer, such a cancer can be detected at least in part using such hypomethylation variable target regions. In some embodiments, hypomethylation variable target regions include one or more genomic regions, where the cfDNA molecules in those regions do not differ in methylation state in cancer subjects relative to cfDNA from healthy subjects, but the presence/increased quantity of hypomethylated cfDNA in those regions is indicative of a particular tissue type (e.g., cancer origin) and is presented as cfDNA with increased apoptosis (e.g. tumor shedding) into circulation.

In some embodiments, hypomethylation variable target regions include repeated elements and/or intergenic regions. In some embodiments, repeated elements include one, two, three, four, or five of LINE1 elements, Alu elements, centromeric tandem repeats, pericentromeric tandem repeats, and/or satellite DNA.

Exemplary specific genomic regions that show cancer-associated hypomethylation include nucleotides 8403565-8953708 and 151104701-151106035 of human chromosome 1. In some embodiments, the hypomethylation variable target regions overlap or comprise one or both of these regions.

In some embodiments, the probes for the epigenetic target region set comprise probes specific for one or more hypomethylation variable target regions. The hypomethylation variable target regions may be any of those set forth above. For example, the probes specific for one or more hypomethylation variable target regions may include probes for regions such as repeated elements, e.g., LINE1 elements, Alu elements, centromeric tandem repeats, pericentromeric tandem repeats, and satellite DNA, and intergenic regions that are ordinarily methylated in healthy cells may show reduced methylation in tumor cells.

In some embodiments, probes specific for hypomethylation variable target regions include probes specific for repeated elements and/or intergenic regions. In some embodiments, probes specific for repeated elements include probes specific for one, two, three, four, or five of LINE1 elements, Alu elements, centromeric tandem repeats, pericentromeric tandem repeats, and/or satellite DNA.

Exemplary probes specific for genomic regions that show cancer-associated hypomethylation include probes specific for nucleotides 8403565-8953708 and/or 151104701-151106035 of human chromosome 1. In some embodiments, the probes specific for hypomethylation variable target regions include probes specific for regions overlapping or including nucleotides 8403565-8953708 and/or 151104701-151106035 of human chromosome

Probes for detecting the panel of regions can include those for detecting genomic regions of interest (hotspot regions) as well as nucleosome-aware probes (e.g., KRAS codons 12 and 13) and may be designed to optimize capture based on analysis of cfDNA coverage and fragment size variation impacted by nucleosome binding patterns and GC sequence composition. Regions used herein can also include non-hotspot regions optimized based on nucleosome positions and GC models. Subjects

In some embodiments, the DNA (e.g., cfDNA) is obtained from a subject having a cancer. In some embodiments, the DNA (e.g., cfDNA) is obtained from a subject suspected of having a cancer. In some embodiments, the DNA (e.g., cfDNA) is obtained from a subject having a tumor. In some embodiments, the DNA (e.g., cfDNA) is obtained from a subject suspected of having a tumor. In some embodiments, the DNA (e.g., cfDNA) is obtained from a subject having neoplasia. In some embodiments, the DNA (e.g., cfDNA) is obtained from a subject suspected of having neoplasia. In some embodiments, the DNA (e.g., cfDNA) is obtained from a subject in remission from a tumor, cancer, or neoplasia (e.g., following chemotherapy, surgical resection, radiation, or a combination thereof). In any of the foregoing embodiments, the cancer, tumor, or neoplasia or suspected cancer, tumor, or neoplasia may be of the lung, colon, rectum, kidney, breast, prostate, or liver. In some embodiments, the cancer, tumor, or neoplasia or suspected cancer, tumor, or neoplasia is of the lung. In some embodiments, the cancer, tumor, or neoplasia or suspected cancer, tumor, or neoplasia is of the colon or rectum. In some embodiments, the cancer, tumor, or neoplasia or suspected cancer, tumor, or neoplasia is of the breast. In some embodiments, the cancer, tumor, or neoplasia or suspected cancer, tumor, or neoplasia is of the prostate. In any of the foregoing embodiments, the subject may be a human subject.

In some embodiments, the sequence-variable target region probe set has a footprint of at least 0.5 kb, e.g., at least 1 kb, at least 2 kb, at least 5 kb, at least 10 kb, at least 20 kb, at least 30 kb, or at least 40 kb. In some embodiments, the epigenetic target region probe set has a footprint in the range of 0.5-100 kb, e.g., 0.5-2 kb, 2-10 kb, 10-20 kb, 20-30 kb, 30-40 kb, 40-50 kb, 50-60 kb, 60-70 kb, 70-80 kb, 80-90 kb, and 90-100 kb.

In some embodiments, the probes specific for the sequence-variable target region set comprise probes specific for target regions from at least 10, 20, 30, or 35 cancer-related genes, such as AKT1, ALK, BRAF, CCND1, CDK2A, CTNNB1, EGFR, ERBB2, ESR1, FGFR1, FGFR2, FGFR3, FOXL2, GATA3, GNA11, GNAQ, GNAS, HRAS, IDH1, IDH2, KIT, KRAS, MED12, MET, MYC, NFE2L2, NRAS, PDGFRA, PIK3CA, PPP2R1A, PTEN, RET, STK11, TP53, and U2AF1.

### Cancer and Other Diseases

The present methods can be used to diagnose presence of conditions, particularly cancer, in a subject, to characterize conditions (e.g., staging cancer or determining heterogeneity of a cancer), monitor response to treatment of a condition, effect prognosis risk of developing a condition or subsequent course of a condition. The present disclosure can also be useful in determining the efficacy of a particular treatment option. Successful treatment options may increase the amount of copy number variation or rare mutations detected in subject's blood if the treatment is successful as more cancers may die and shed DNA. In other examples, this may not occur. In another example, perhaps certain treatment options may be correlated with genetic profiles of cancers over time. This correlation may be useful in selecting a therapy.

Additionally, if a cancer is observed to be in remission after treatment, the present methods can be used to monitor residual disease or recurrence of disease.

In some embodiments, the methods and systems disclosed herein may be used to identify customized or targeted therapies to treat a given disease or condition in patients based on the classification of a nucleic acid variant as being of somatic or germline origin. Typically, the disease under consideration is a type of cancer. Non-limiting examples of such cancers include biliary tract cancer, bladder cancer, transitional cell carcinoma, urothelial carcinoma, brain cancer, gliomas, astrocytomas, breast carcinoma, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, Wilms tumor, leukemia, acute lymphocytic leukemia (ALL), acute myeloid leukemia (AML), chronic lymphocytic leukemia (CLL), chronic myeloid leukemia (CML), chronic myelomonocytic leukemia (CMML), liver cancer, liver carcinoma, hepatoma, hepatocellular carcinoma, cholangiocarcinoma, hepatoblastoma, Lung cancer, non-small cell lung cancer (NSCLC), mesothelioma, B-cell lymphomas, non-Hodgkin lymphoma, diffuse large B-cell lymphoma, Mantle cell lymphoma, T cell lymphomas, non-Hodgkin lymphoma, precursor T-lymphoblastic lymphoma/leukemia, peripheral T cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas. Prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, or uterine sarcoma. Type and/or stage of cancer can be detected from genetic variations including mutations, rare mutations, indels, copy number variations, transversions, translocations, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, and abnormal changes in nucleic acid 5-methylcytosine.

Genetic data can also be used for characterizing a specific form of cancer. Cancers are often heterogeneous in both composition and staging. Genetic profile data may allow characterization of specific sub-types of cancer that may be important in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers can progress to become more aggressive and genetically unstable. Other cancers may remain benign, inactive or dormant. The system and methods of this disclosure may be useful in determining disease progression.

Further, the methods of the disclosure may be used to characterize the heterogeneity of an abnormal condition in a subject. Such methods can include, e.g., generating a genetic profile of extracellular polynucleotides derived from the subject, wherein the genetic profile includes a plurality of data resulting from copy number variation and rare mutation analyses. In some embodiments, an abnormal condition is cancer. In some embodiments, the abnormal condition may be one resulting in a heterogeneous genomic population. In the example of cancer, some tumors are known to comprise tumor cells in different stages of the cancer. In other examples, heterogeneity may comprise multiple foci of disease. Again, in the example of cancer, there may be multiple tumor foci, perhaps where one or more foci are the result of metastases that have spread from a primary site.

The present methods can be used to generate our profile, fingerprint or set of data that is a summation of genetic information derived from different cells in a heterogeneous disease. This set of data may comprise copy number variation, epigenetic variation, and mutation analyses alone or in combination.

The present methods can be used to diagnose, prognose, monitor or observe cancers, or other diseases. In some embodiments, the methods herein do not involve the diagnosing, prognosing or monitoring a fetus and as such are not directed to non-invasive prenatal testing. In other embodiments, these methodologies may be employed in a pregnant subject to diagnose, prognose, monitor or observe cancers or other diseases in an unborn subject whose DNA and other polynucleotides may co-circulate with maternal molecules.

Non-limiting examples of other genetic-based diseases, disorders, or conditions that are optionally evaluated using the methods and systems disclosed herein include achondroplasia, alpha-1 antitrypsin deficiency, antiphospholipid syndrome, autism, autosomal dominant polycystic kidney disease, Charcot-Marie-Tooth (CMT), cri du chat, Crohn's disease, cystic fibrosis, Dercum disease, down syndrome, Duane syndrome, Duchenne muscular dystrophy, Factor V Leiden thrombophilia, familial hypercholesterolemia, familial Mediterranean fever, fragile X syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency (SCID), sickle cell disease, spinal muscular atrophy, Tay-Sachs, thalassemia, trimethylaminuria, Turner syndrome, velocardiofacial syndrome, WAGR syndrome, Wilson disease, or the like.

In some embodiments, a method described herein includes detecting a presence or absence of DNA originating or derived from a tumor cell at a preselected timepoint following a previous cancer treatment of a subject previously diagnosed with cancer using a set of sequence information obtained as described herein. The method may further comprise determining a cancer recurrence score that is indicative of the presence or absence of the DNA originating or derived from the tumor cell for the test subject. Where a cancer recurrence score is determined, it may further be used to determine a cancer recurrence status. The cancer recurrence status may be at risk for cancer recurrence, e.g., when the cancer recurrence score is above a predetermined threshold. The cancer recurrence status may be at low or lower risk for cancer recurrence, e.g., when the cancer recurrence score is above a predetermined threshold. In particular embodiments, a cancer recurrence score equal to the predetermined threshold may result in a cancer recurrence status of either at risk for cancer recurrence or at low or lower risk for cancer recurrence.

In some embodiments, a cancer recurrence score is compared with a predetermined cancer recurrence threshold, and the test subject is classified as a candidate for a subsequent cancer treatment when the cancer recurrence score is above the cancer recurrence threshold or not a candidate for therapy when the cancer recurrence score is below the cancer recurrence threshold. In particular embodiments, a cancer recurrence score equal to the cancer recurrence threshold may result in classification as either a candidate for a subsequent cancer treatment or not a candidate for therapy.

The methods discussed above may further comprise any compatible feature or features set forth elsewhere herein, including in the section regarding methods of determining a risk of cancer recurrence in a test subject and/or classifying a test subject as being a candidate for a subsequent cancer treatment.

### Therapies and Related Administration

In certain embodiments, the methods disclosed herein relate to identifying and administering customized therapies to patients given the status of a nucleic acid variant as being of somatic or germline origin. In some embodiments, essentially any cancer therapy (e.g., surgical therapy, radiation therapy, chemotherapy, and/or the like) may be included as part of these methods. Typically, customized therapies include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In certain embodiments, the status of a nucleic acid variant from a sample from a subject as being of somatic or germline origin may be compared with a database of comparator results from a reference population to identify customized or targeted therapies for that subject. Typically, the reference population includes patients with the same cancer or disease type as the test subject and/or patients who are receiving, or who have received, the same therapy as the test subject. A customized or targeted therapy (or therapies) may be identified when the nucleic variant and the comparator results satisfy certain classification criteria (e.g., are a substantial or an approximate match).

In certain embodiments, the customized therapies described herein are typically administered parenterally (e.g., intravenously or subcutaneously). Pharmaceutical compositions containing an immunotherapeutic agent are typically administered intravenously. Certain therapeutic agents are administered orally. However, customized therapies (e.g., immunotherapeutic agents, etc.) may also be administered by methods such as, for example, buccal, sublingual, rectal, vaginal, intraurethral, topical, intraocular, intranasal, and/or intraauricular, which administration may include tablets, capsules, granules, aqueous suspensions, gels, sprays, suppositories, salves, ointments, or the like.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it should be understood that all aspects of the invention are not limited to the specific depictions, configurations or relative proportions set forth herein which depend upon a variety of conditions and variables. It should be understood that various alternatives to the embodiments of the disclosure described herein may be employed in practicing the invention. It is therefore contemplated that the disclosure shall also cover any such alternatives, modifications, variations or equivalents. It is intended that the following claims define the scope of the invention and that methods and structures within the scope of these claims and their equivalents be covered thereby.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, systems, computer readable media, and/or component features, steps, elements, or other aspects thereof can be used in various combinations.

Detection of a biomarker may indicate the presence of one or more cancers. Detection may indicate presence of a cancer selected from the group including ovarian cancer, pancreatic cancer, breast cancer, colorectal cancer, non-small cell lung carcinoma (e.g., squamous cell carcinoma, or adenocarcinoma) or any other cancer. Detection may indicate the presence of any cancer selected from the group including ovarian cancer, pancreatic cancer, breast cancer, colorectal cancer, non-small cell lung carcinoma (squamous cell or adenocarcinoma) or any other cancer. Detection may indicate the presence of any of a plurality of cancers selected from the group including ovarian cancer, pancreatic cancer, breast cancer, colorectal cancer and non-small cell lung carcinoma (squamous cell or adenocarcinoma), or any other cancer. Detection may indicate presence of one or more of any of the cancers mentioned in this application.

One or more cancers may exhibit a biomarker in at least one exon in the panel. One or more cancers selected from the group including ovarian cancer, pancreatic cancer, breast cancer, colorectal cancer, non-small cell lung carcinoma (squamous cell or adenocarcinoma), or any other cancer, each exhibit a biomarker in at least one exon in the panel. Each of at least 3 of the cancers may exhibit a biomarker in at least one exon in the panel. Each of at least 4 of the cancers may exhibit a biomarker in at least one exon in the panel. Each of at least 5 of the cancers may exhibit a biomarker in at least one exon in the panel. Each of at least 8 of the cancers may exhibit a biomarker in at least one exon in the panel. Each of at least 10 of the cancers may exhibit a biomarker in at least one exon in the panel. All of the cancers may exhibit a biomarker in at least one exon in the panel.

If a subject has a cancer, the subject may exhibit a biomarker in at least one exon or gene in the panel. At least 85% of subjects having a cancer may exhibit a biomarker in at least one exon or gene in the panel. At least 90%, of subjects having a cancer may exhibit a biomarker in at least one exon or gene in the panel. At least 92% of subjects having a cancer may exhibit a biomarker in at least one exon or gene in the panel. At least 95% of subjects having a cancer may exhibit a biomarker in at least one exon or gene in the panel. At least 96% of subjects having a cancer may exhibit a biomarker in at least one exon or gene in the panel. At least 97% of subjects having a cancer may exhibit a biomarker in at least one exon or gene in the panel. At least 98% of subjects having a cancer may exhibit a biomarker in at least one exon or gene in the panel. At least 99% of subjects having a cancer may exhibit a biomarker in at least one exon or gene in the panel. At least 99.5% of subjects having a cancer may exhibit a biomarker in at least one exon or gene in the panel.

If a subject has a cancer, the subject may exhibit a biomarker in at least one region in the panel. At least 85% of subjects having a cancer may exhibit a biomarker in at least one region in the panel. At least 90%, of subjects having a cancer may exhibit a biomarker in at least one region in the panel. At least 92% of subjects having a cancer may exhibit a biomarker in at least one region in the panel. At least 95% of subjects having a cancer may exhibit a biomarker in at least one region in the panel. At least 96% of subjects having a cancer may exhibit a biomarker in at least one region in the panel. At least 97% of subjects having a cancer may exhibit a biomarker in at least one region in the panel. At least 98% of subjects having a cancer may exhibit a biomarker in at least one region in the panel. At least 99% of subjects having a cancer may exhibit a biomarker in at least one region in the panel. At least 99.5% of subjects having a cancer may exhibit a biomarker in at least one region in the panel.

Detection may be performed with a high sensitivity and/or a high specificity. Sensitivity can refer to a measure of the proportion of positives that are correctly identified as such. In some cases, sensitivity refers to the percentage of all existing biomarkers that are detected. In some cases, sensitivity refers to the percentage of sick people who are correctly identified as having certain disease. Specificity can refer to a measure of the proportion of negatives that are correctly identified as such. In some cases, specificity refers to the proportion of unaltered bases which are correctly identified. In some cases, specificity refers to the percentage of healthy people who are correctly identified as not having certain disease. The non-unique tagging method described previously significantly increases specificity of detection by reducing noise generated by amplification and sequencing errors, which reduces frequency of false positives. Detection may be performed with a sensitivity of at least 95%, 97%, 98%, 99%, 99.5%, or 99.9% and/or a specificity of at least 80%, 90%, 95%, 97%, 98% or 99%. Detection may be performed with a sensitivity of at least 90%, 95%, 97%, 98%, 99%, 99.5%, 99.6%, 99.98%, 99.9% or 99.95%. Detection may be performed with a specificity of at least 90%, 95%, 97%, 98%, 99%, 99.5%, 99.6%, 99.98%, 99.9% or 99.95%. Detection may be performed with a specificity of at least 70% and a sensitivity of at least 70%, a specificity of at least 75% and a sensitivity of at least 75%, a specificity of at least 80% and a sensitivity of at least 80%, a specificity of at least 85% and a sensitivity of at least 85%, a specificity of at least 90% and a sensitivity of at least 90%, a specificity of at least 95% and a sensitivity of at least 95%, a specificity of at least 96% and a sensitivity of at least 96%, a specificity of at least 97% and a sensitivity of at least 97%, a specificity of at least 98% and a sensitivity of at least 98%, a specificity of at least 99% and a sensitivity of at least 99%, or a specificity of 100% a sensitivity of 100%. In some cases, the methods can detect a biomarker at a sensitivity of sensitivity of about 80% or greater. In some cases, the methods can detect a biomarker at a sensitivity of sensitivity of about 95% or greater. In some cases, the methods can detect a biomarker at a sensitivity of sensitivity of about 80% or greater, and a sensitivity of sensitivity of about 95% or greater.

Detection may be highly accurate. Accuracy may apply to the identification of biomarkers in cell free DNA, and/or to the diagnosis of cancer. Statistical tools, such as co-variate analysis described above, may be used to increase and/or measure accuracy. The methods can detect a biomarker at an accuracy of at least 80%, 90%, 95%, 97%, 98% or 99%, 99.5%, 99.6%, 99.98%, 99.9%, or 99.95%. In some cases, the methods can detect a biomarker at an accuracy of at least 95% or greater.

In some cases, the cancer treatment includes, without limitation, atezolizumab (Tecentriq), imatinib, gefatinib, afatinib, dacomitinib, sunitinib, sorafenib, vandetanib, brivanib, cabozantib, neratinib, tivantinib, bevacizumab, cixutumumab, dalotuzumab, figitumumab, rilotumumab, onartuzumab, ganitumab, ramucirumab, ridaforolimus, tensirolimus, everolimus, BMS-690514, BMS-754807, EMD 525797, GDC-0973, GDC-0941, MK-2206, AZD6244, GSK1120212, PX-866, XL821, IMC-A12, MM-121, PF-02341066, RG7160, and Sym004. Antibodies suitable for use as anti-EGFR therapy include cetuximab (Trade Name: Erbitux) and panitumumab (Trade Name: Vectibex). In some cases. In some cases, the cancer treatment includes EGFR tyrosine kinase inhibitors such as gefitinib (Trade Name: Iressa), erlotinib (Trade Name: Tarceva), lapatinib, canertinib, and cetuximab.

In some instances, therapies may be used in combination, such as an anti-EGFR therapy and an anti-EGFR therapy. Anti-EGFR therapy may be used in combination with any combination of chemotherapeutic agents or chemotherapeutic regimens, for example, FOLFOX (fluorouracil [5-FU]/leucovorin/oxaliplatin), FOLFIRI (5-FU/leucovorin/irinotecan), and the like.

In some embodiments, the therapy includes an epigenetic regulator, including HAT, HDAC inhibitors as examples. Other examples including, EP015666, LLY-283, JNJ-64619178, BRD0639, AMG193, TNG908, SCR-6920, PRT543, PRT811, MRTX1719, cycloleucine, aminobicycle-hexane-carboxcyclic acid, FIDAS agents, PF-9366, AGR-25696, AG-270, Compound 28, IDE397. Other examples include agents described in Bray et al., Front. Onco. 2023, which is fully incorporated by reference herein.

In some embodiments, the therapy includes paclitaxel (chemotherapeutic drug), ipatasertib (AKT inhibitor), PI3K-Beta inhibitor, AZD8186, docetaxel, tyrosine kinase inhibitor, pazopanib, mTOR inhibitor, everolimus (NCT01430572), PI3K-Beta inhibitor GSK2636771, and immunotherapy, pembrolizumab (NCT03131908), tastuzumab. Other examples include agents described in Ertay et al., Genes and Diseases. 2023, and Dillon and Miller Curr Drug Targets 2015, each of which is fully incorporated by reference herein.

In some aspects, a cancer treatment is administered to a subject. In some cases, the cancer treatment is administered in combination another therapy, such as a non-anti-EGFR therapy with anti-EGFR therapy.

In various embodiments, cancer treatments can include ipilimumab (Yervoy), a CTLA4 inhibitor applied based on PD-L1 protein expression, also tremelimumab (Imjuno); Nivolumab (Opdivo) is PD-1 inhibitor that can be utilized in combination with ipilimumab, optionally included platinum; Other PD-1 inhibitors include pembrolizumab (Keytruda), cemiplimab-rwlc (Libtayo), durvalumab (Imfinzi) which are utilized in unresectable NSCLC. Further information is found in Basudan Clin Pract. 2023 February; 13 (1): 22-40, Guo et al. Front Oncol 2022 Aug. 11; 12; and Meng et al., Cell Death Dis 15, 3 (2024), each of which is fully incorporated by referenced herein. In various aspects, the cancer treatment is administered to the subject.

### EXAMPLES

### Example 1—Attributes of the Methylation Data

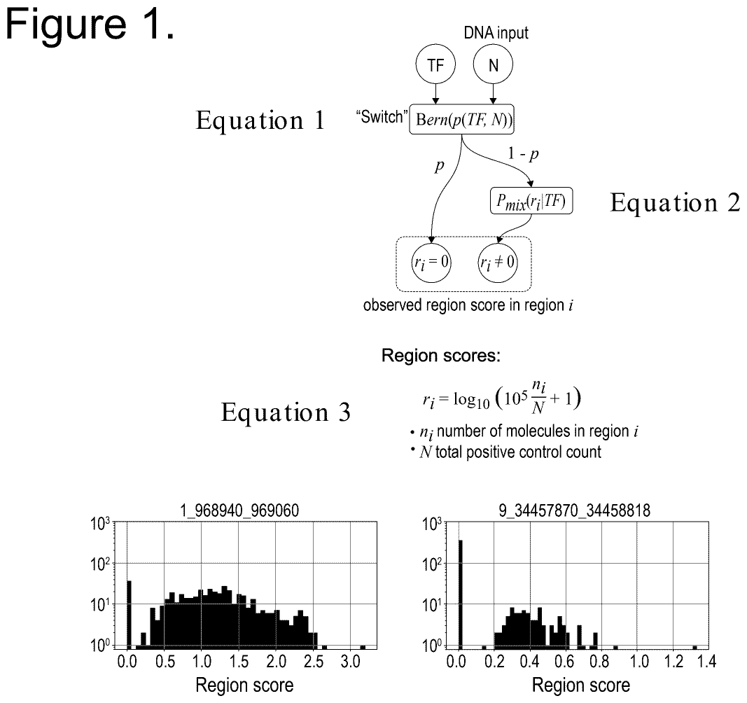

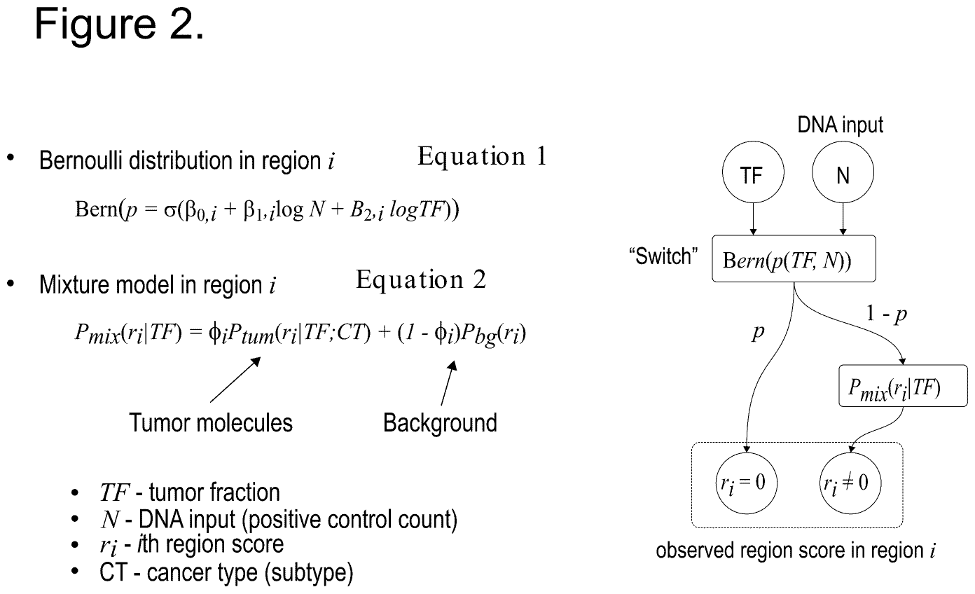

Methylation data can be characterized as possessing sparsity as a result of the regions that can turn on/off sporadically. The degree of sparsity of the signal correlates with the DNA input and TF. To take this biological activity into account for improving the accuracy of methylation measurements, one can taken into account, a region score (molecule count in a region) that depends on the TF and incorporate the parameters of observed molecules that are a mixture of normal molecules (background) and tumor molecules. For example, one can establish a model which combines both a mixture component involving normal and tumor molecules as features, and a Bernoulli distribution to account for probabilistic testing of sporadic silencing and activation of methylation, as shown in FIGS. 1 and 2.

### Example 2—Study Design

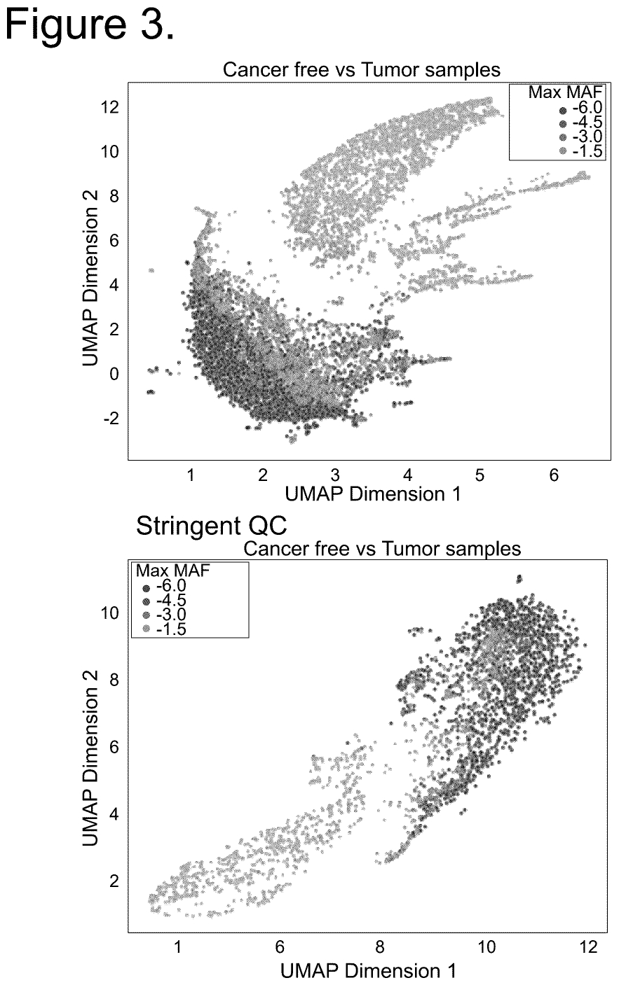

Here, the Inventors established the generative probabilistic model (FIGS. 1 and 2) which incorporates the attributes and features of methylation data. Starting with 23,869 samples, sub-sets included cancer free samples (No non-synonymous SNV/Indels in reportable genes), tumor BRCA samples (found to have non-synonymous SNVs/Indels in reportable genes). This data included fragment size distribution is not far from the average distribution of >23,000 samples (Eucl.dist). Characteristics of each of these data sets is shown in FIG. 3.

### Example 3—Exemplary Applications of Generative Probabilistic Modeling

Given that the above features of methylation data are largely universal, the described model therefore finds wide extensibility for increasing informative signal data in various settings.

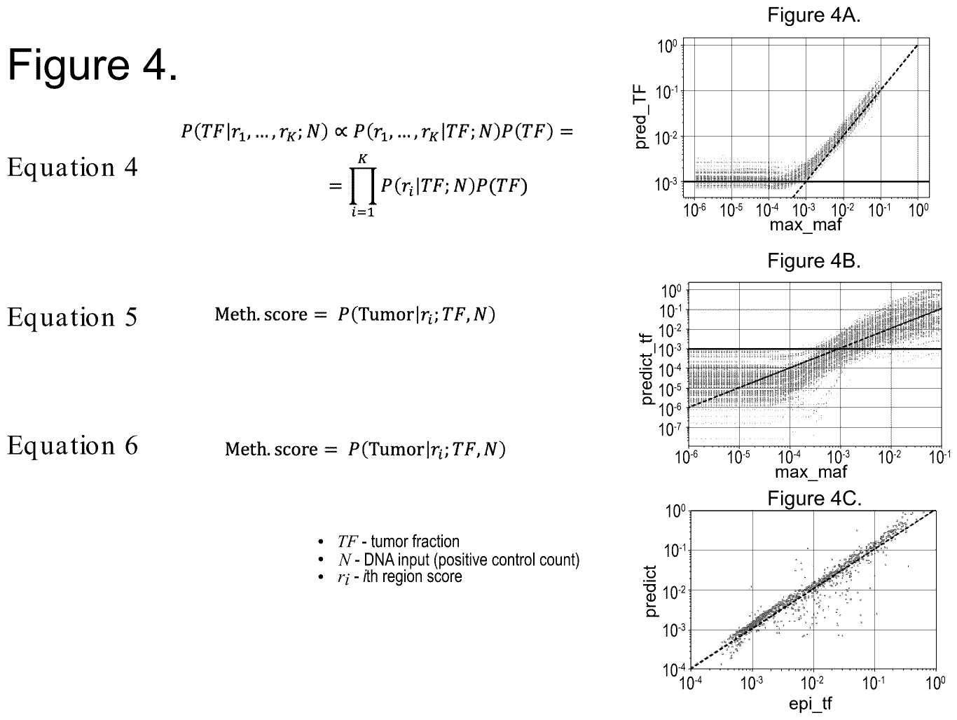

Various examples of applications include measuring tumor fraction by inferring unknown tumor fraction as a latent variable (Equation 4), normalized methylation score (for cancer subtyping) by using the probability of tumor molecules present in a genomic region given a region score (Equation 5), tumor fraction and DNA input of a sample, or predicting the likelihood of different cancer subtypes (Equation 6) with each shown in FIG. 4. Performance characteristics are shown in FIG. 4A (mixture component), 4B (Bernoulli) and 4C for characterizing BRCA samples.

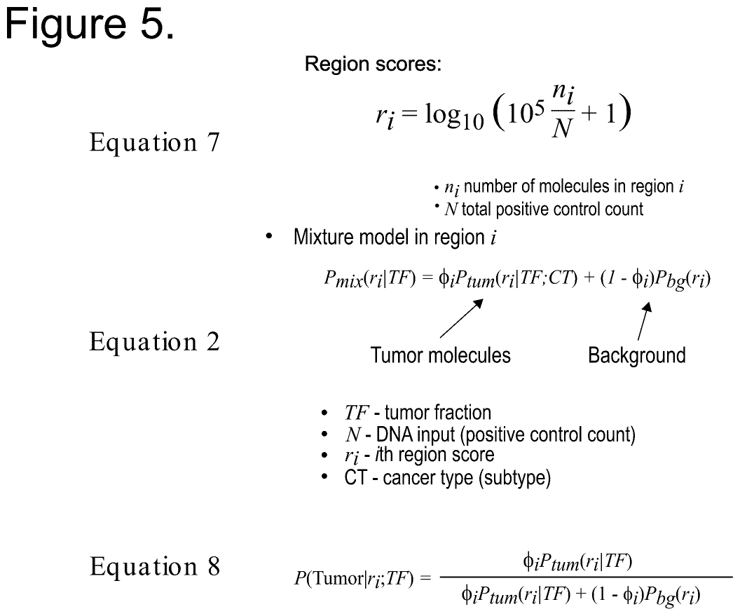

FIG. 5 shows exemplary data of probability of tumor molecules present in a genomic region, which begins with region score (Equation 7) followed by fitting of background distribution to normal cancer free samples, then fitting the mixture to tumor BRCA samples (Equation 2). Thereafter, a test sample can be evaluated for the probability of tumor molecules in the region given the DNA input (total pos. cntrl.) and tumor fraction, having fitted distributions between normal and cancer samples (Equation 8).

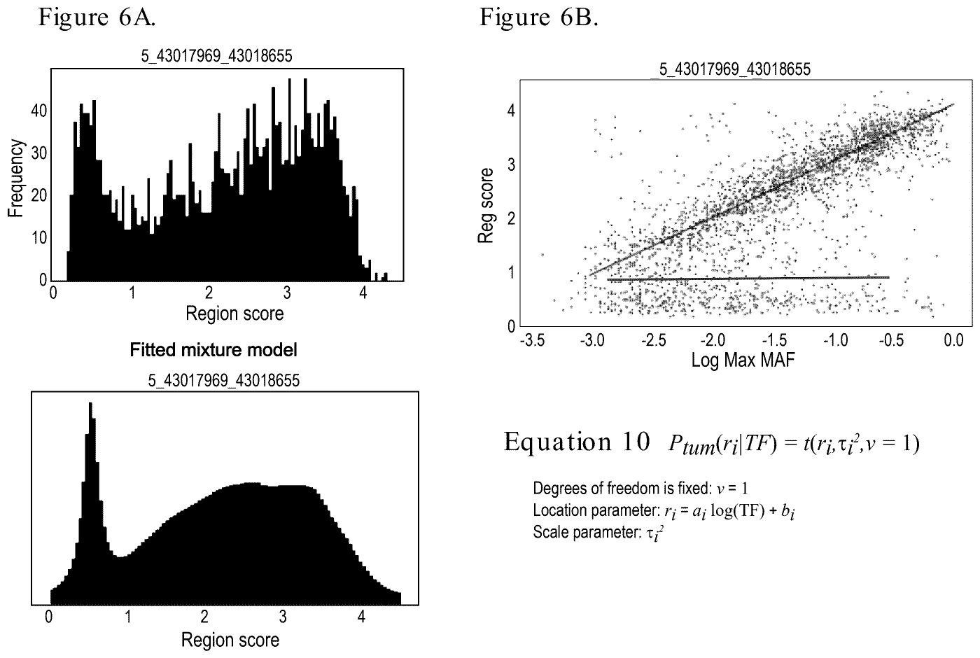

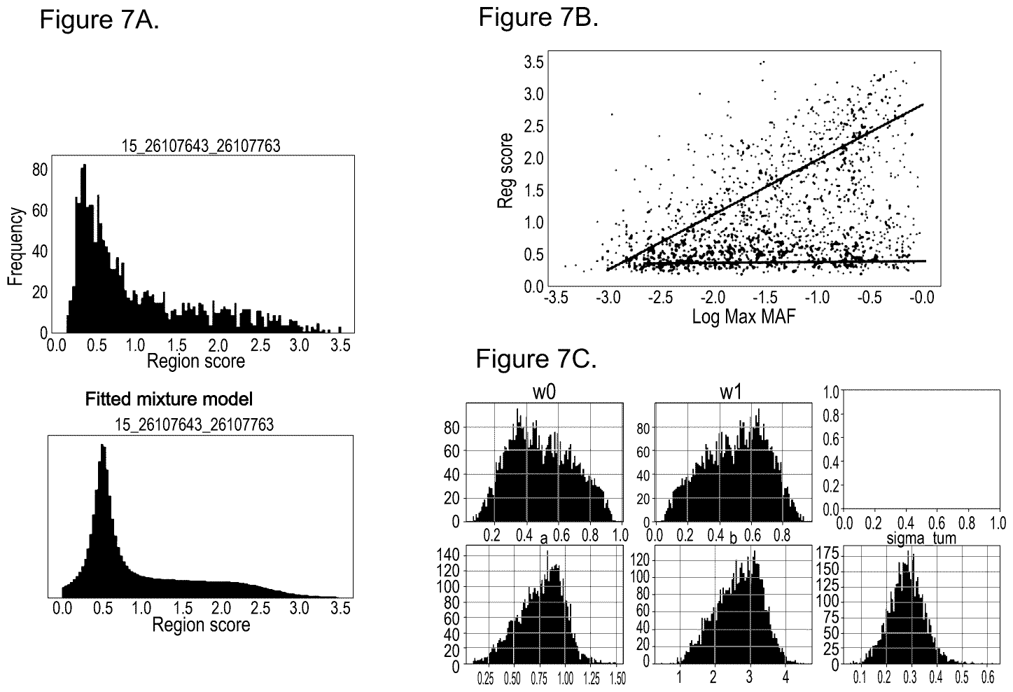

Illustrative examples of this process are shown in FIGS. 6 and 7. In some embodiments, one can further apply a filter to exclude the regions where the fit quality is poor.

### Example 4—Probabilistic Modeling of Methylation Signal

Described herein is use of the described generative probabilistic modeling method that allows determination of the likelihood of tumor molecule presence in a genomic region for a given TF and DNA input of the sample.

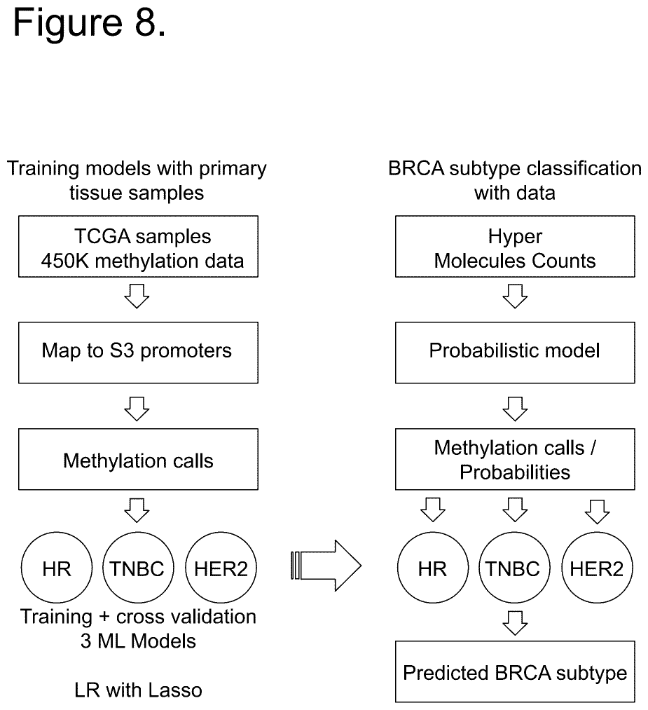

For performance comparison, described are two approaches to BRCA subtyping, illustrated in FIG. 8. A first approach includes transferring the subtype prediction models between different technologies to train three models (HR/HER2/TNBC) using 450K methylation calls data from TCGA primary tissue samples and applying these subtype prediction models to methylation calls for subtype prediction. A second approach is to training the models (LR+Lasso) directly on epigenomic methylation data (samples with known subtypes) and evaluate the performance using cross validation.

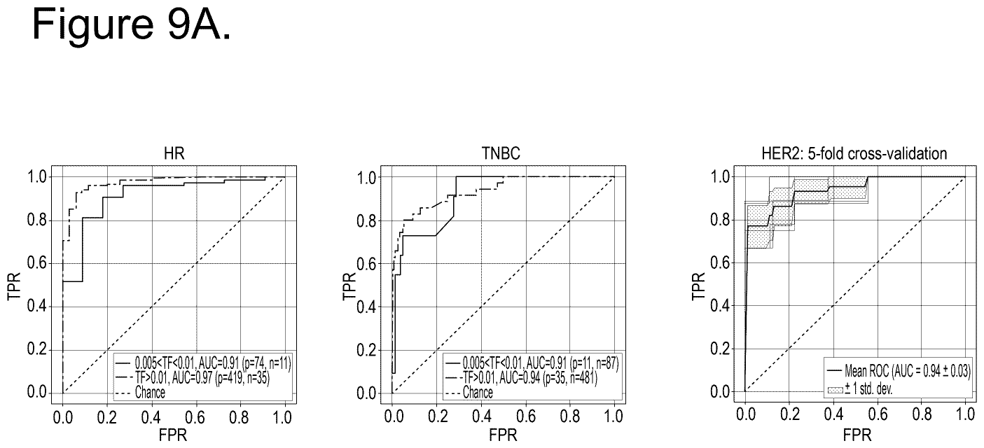

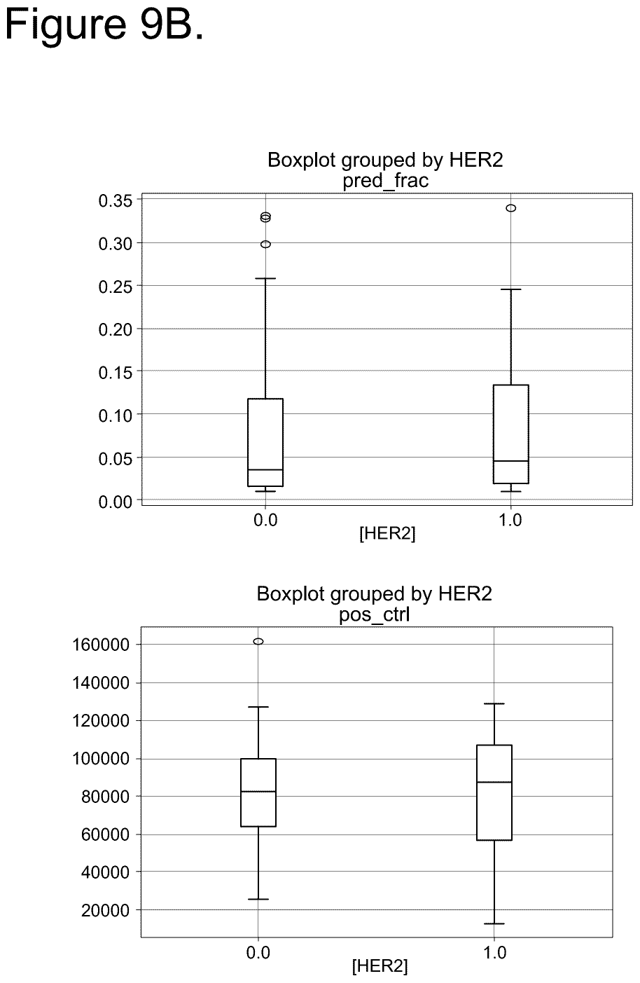

Results are shown in Tables 1 and 2. And further results are provided in FIGS. 9A and 9B as shown, the described generative probabilistic method provides excellent determination of subtype accuracy, at least comparable and in some instances superior to direct training on sample followed by cross validation.

TABLE 1
Cohorts: HR+/HER−, TNBC

| Tumor | HR | | TNBC | |
|---|---|---|---|---|
| fraction | PPA (95% CI) | NPA (95% CI) | PPA (95% CI) | NPA (95% CI) |
| [0.005, 0.01] | 0.919+/−0.062 | 0.727+/−0.263 | 0.727+/−0.263 | 0.885+/−0.067 |
| [0.01, 1] | 0.895+/−0.029 | 0.943+/−0.77 | 0.829+/−0.125 | 0.896+/−0.027 |

TABLE 2
Cohort of 200 BRCA samples: HR+/HER2−, HR+/HER2+, HR−/HER2+

| Tumor | HR | | HER2* | |
|---|---|---|---|---|
| fraction | PPA (95% CI) | NPA (95% CI) | PPA (95% CI) | NPA (95% CI) |
| [0.005, 0.01] | 0.727+/−0.263 | 0.824+/−0.181 | 0.571+/−0.367 | 0.7+/−0.284 |
| [0.01, 1] | 0.824+/−0.128 | 0.915+/−0.056 | 0.814+−0.249 | 0.903+/−0.108 |

### Example 5—Methylation Signature for SCLC Transition

In other embodiments, one can utilize the above methods for identifying small cell lung cancer (SCLC) transformation. SCLC transformation represents a mechanism of resistance to epidermal growth factor receptor tyrosine kinase inhibitors (EGFR-TKIs) in EGFR mutated non-small cell lung cancer (NSCLC) cases, which dramatically impacts patients' prognosis due to high refractoriness to conventional treatments.

TABLE 3
Small cell lung cancer (SCLC) transformation represents
a mechanism of resistance to epidermal growth factor
receptor tyrosine kinase inhibitors

| NSCLC patients | SCLC transition | non transition |
|---|---|---|
| respondents to | 0 | 0.55 |
| TKI treatment | | |
| non respondents | 0.15 | 0.35 |

### Example 6—Methylation Signature for SCLC Transition

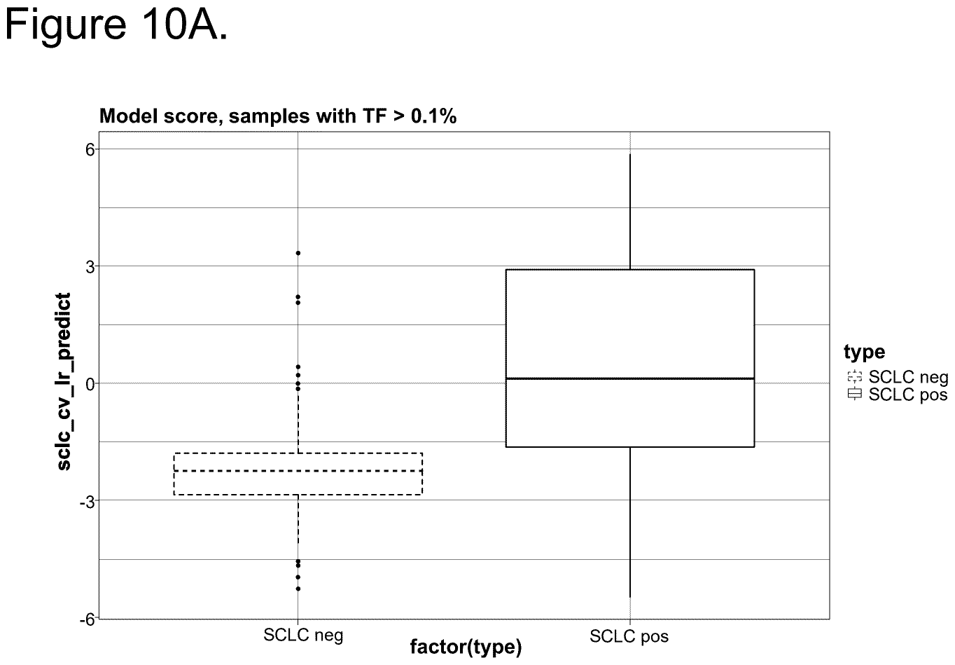

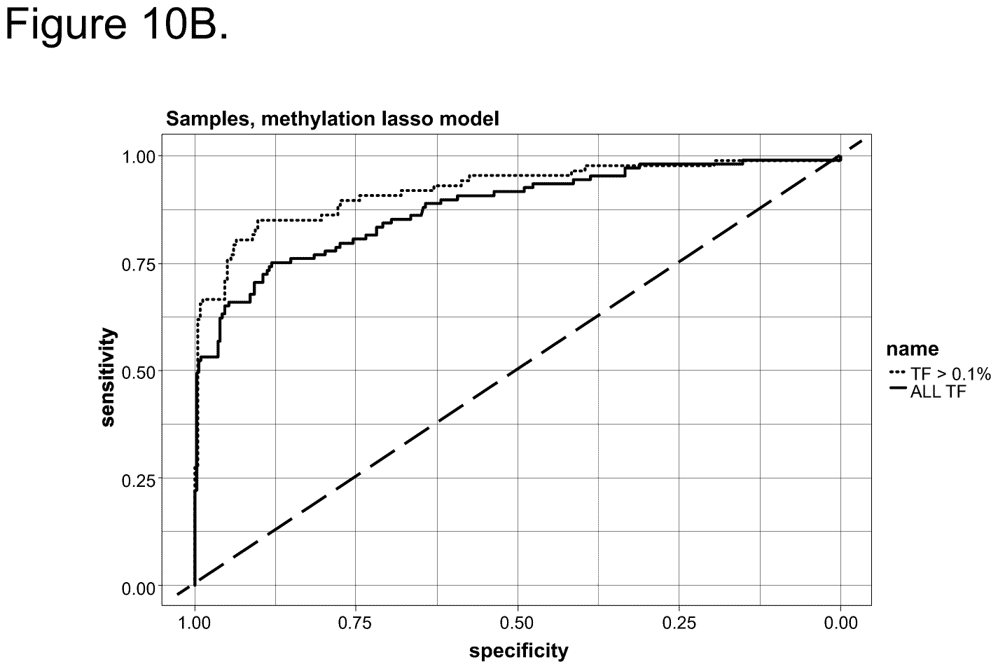

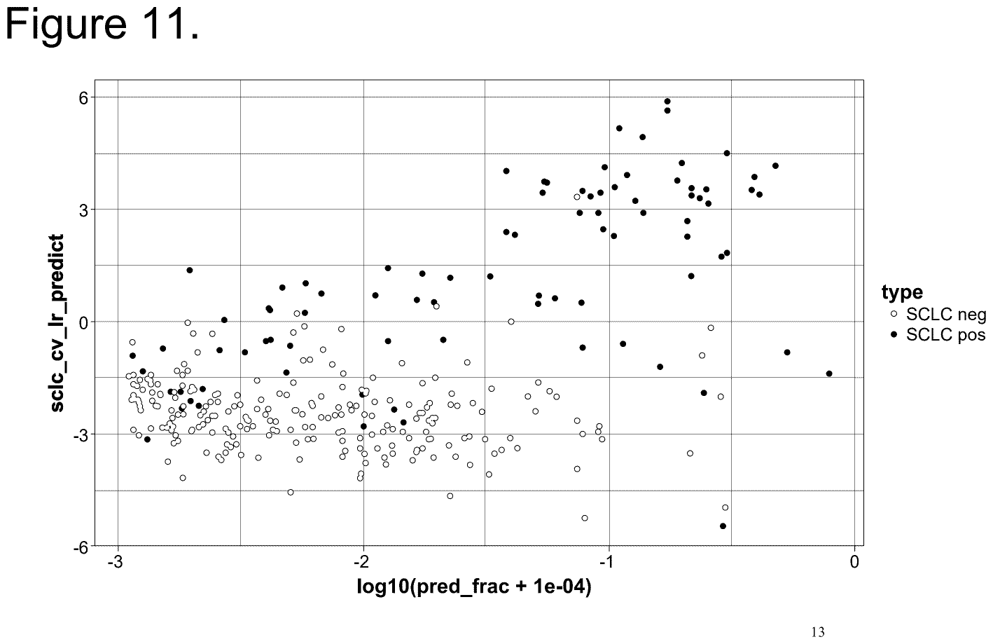

Without being bound by particular theory, various hypotheses have been used to explain the pathogenesis of SCLC transformation. Although SCLC transformation is not common in clinical practice, it has been repeatedly identified in many small patient series and case reports. It usually occurs in epidermal growth factor receptor (EGFR) mutant lung adenocarcinoma after treatment with tyrosine kinase inhibitors (TKIs). SCLC transformation can also occur in anaplastic lymphoma kinase (ALK)-positive lung cancer after treatment with ALK inhibitors and in wild-type EGFR or ALK NSCLC treated with immunotherapy. Chemotherapy was previously used to treat transformed SCLC, yet it is associated with an unsatisfactory prognosis Results are shown in FIGS. 10 and 11.

### Example 7—Methylation Signature for Prostate Cancer Patients Who have had SCLC Transformation

Determination of signature includes comparison of cohorts at diagnosis of metastatic lung adenocarcinoma. For example, one can identify samples labeled as lung adenocarcinoma in 1st timepoint, with activating EGFR mutation (and RB1 loss as a comparator cohort) treated with EGFRi (these are most prone to transformation). Similarly, one can compare patients who have T790M or C797S for progression on EGFRi as the first sample for lung adeno. Alternatively, measurements of methylation are at any number of subsequent timepoints, including the timepoint prior to switching to carbo etoposide

### Example 8—Methylation Signature for NEPC Transformation

In other embodiments, one can utilize the above methods for subtyping of Neuroendocrine prostate cancer (NEPC), which is a subtype of prostate cancer that most commonly arising in later stage prostate cancer in connection with treatment resistance. NEPC commonly develops as a result of lineage plasticity whereby prostate cancer cells adopt alternative lineage program, including evading therapy. Androgen receptor (AR) signaling decreases as tumors progress from a prostate adenocarcinoma to a NEPC histology, including downregulation of AR, PSA, and PSMA expression in tumors. While genomic analyses from patient biopsies combined with preclinical modeling has shown loss of tumor suppressors RB1 and TP53 as facilitating lineage plasticity. Activation of oncogenic drivers combined and/or significant epigenetic changes (e.g., EZH2 overexpression, DNA methylation) further drive tumor proliferation, further shown by expression of downstream neuronal and neuroendocrine lineage pathways involving pioneer and lineage determinant transcription factors.

### Example 9—Methylation Signature for Prostate Cancer Patients Who have had Neuroendocrine Transformation

The aforementioned techniques can also be used for identifying methylation signatures capable of determining presence, absence, and/or likelihood of neuroendocrine transformation, including in metastatic neuroendocrine-transformed prostate cancer (NEPC). Determination of methylation signature can be made in comparison of cohorts of prostate cancer patients with samples collected pre- and/or post-neuroendocrine transformation. Thereafter, the methylation signature can serve to differentiate between NEPC and prostate adenocarcinoma via a methylation-based signature

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it should be understood that all aspects of the invention are not limited to the specific depictions, configurations or relative proportions set forth herein which depend upon a variety of conditions and variables. It should be understood that various alternatives to the embodiments of the disclosure described herein may be employed in practicing the invention. It is therefore contemplated that the disclosure shall also cover any such alternatives, modifications, variations or equivalents. It is intended that the following claims define the scope of the invention and that methods and structures within the scope of these claims and their equivalents be covered thereby.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, systems, computer readable media, and/or component features, steps, elements, or other aspects thereof can be used in various combinations.

1. A method, comprising:
obtaining methylation data from a sample;
generating a model based on the methylation data, wherein the model comprises at least two parameters and a probabilistic distribution for each of a plurality of sites;
transforming the methylation data based on the generated model; and
determining at least one quantitative metric of the transformed methylation data.

2. The method of claim 1, wherein the at least two parameters comprise a molecule count, mixture component, or both.

3. The method of claim 2, wherein the molecule count comprises a region score.

4. The method of claim 2, wherein the mixture component comprises a measurement of tumor and normal molecules.

5. The method of claim 1, wherein the probabilistic distribution comprises a Bernoulli distribution.

6. The method of claim 1, wherein the methylation data is generated by detecting methylation in at least one of a plurality of sites.

7. The method of claim 1, wherein the plurality of sites are obtained from a sample.

8. The method of claim 1, wherein determining at least one quantitative metric of the transformed methylation data characterizes a sample.

9. The method of claim 8, wherein characterizing the sample comprises determining the sample is derived from one or more subtypes.

10. The method of claim 9, wherein the one or more subtypes are selected from the group consisting of: lung adenocarcinomas (LUAD), lung squamous cell carcinomas (LUSC), small cell lung cancer (SCLC) and/or non-small cell lung cancer (NSCLC).

11. The method of claim 9, wherein the one or more subtypes are selected from the group consisting of: HR, HER2, and TNBC.

12. The method of claim 8, wherein characterizing the sample comprises determining transition, transformation or other alteration of a cancer disease phenotype.

13. The method of claim 1, further comprising obtaining a sample.

14. The method of claim 1, further comprising having obtained a sample.

15. The method of claim 8, further comprising recommending and/or selecting a treatment based on the characterization of the sample.

16. The method of claim 8, further comprising administering a treatment based on the characterization of the sample.

17. The method of claim 1, wherein the model comprises one or more of Equation 1, 2, 3, 4, 5, 6, 7, and 8.

18. The method of claim 16, wherein the treatment comprises one or more therapeutic agents selected from the group consisting of: cisplatin, carboplatin, gemcitabine, taxanes, pemetrexed, VEGFR inhibitor, bevacizumab, EGFR inhibitor, and erlotinib.

19. The method of claim 1, wherein the sample comprises cell-free DNA.

20. The method of claim 1, further comprising: diagnosing a subject, or prognosing a subject for one or more outcomes.

21. (canceled)

22. A system configured to perform the method of claim 1.

23. A computer readable apparatus comprising a storage medium, the storage medium comprising a plurality of instructions configured to, when executed by one or more processors, perform the method of claim 1.
